(19) United States
(12) Patent Application Publication
(10) Pub. No.: US 2023/0257432 A1
(43) Pub. Date: Aug. 17, 2023

(54) COMPOSITIONS AND METHODS FOR SCREENING 4R TAU TARGETING AGENTS

(71) Applicant: Regeneron Pharmaceuticals, Inc., Tarrytown, NY (US)

(72) Inventors: Jeffery Haines, New York, NY (US); Keith Crosby, Pleasantville, NY (US)

(21) Appl. No.: 18/167,517

(22) Filed: Feb. 10, 2023

Related U.S. Application Data
(60) Provisional application No. 63/309,055, filed on Feb. 11, 2022.

(51) Int. Cl. C07K 14/47; C12N 15/86; C12N 5/071; C12N 15/85

(52) U.S. Cl. CPC C07K 14/4711; C12N 15/86; C12N 5/0686; C12N 15/8509; C07K 2319/60; C12N 2750/00043; A01K 2227/105; A01K 2217/075

(57) ABSTRACT

Tau reporter compositions, tau reporter cells, and tau reporter animals are provided that comprise a four-repeat (4R) tau isoform linked to a first reporter protein and a three-repeat (3R) tau isoform linked to a second reporter protein that is different from the first reporter protein. Methods are provided for making such tau reporter cells and tau reporter animals and for using such tau reporter cells and tau reporter animals for assessing the activity of tau-targeting reagents.

### CROSS-REFERENCE TO RELATED APPLICATIONS

This application claims the benefit of U.S. Application 63/309,055, filed Feb. 11, 2022, which is herein incorporated by reference in its entirety for all purposes.

### REFERENCE TO A SEQUENCE LISTING SUBMITTED AS AN XML FILE VIA EFS WEB

The Sequence Listing written in file 057766-590207.xml is 123 kilobytes, was created on Feb. 3, 2023, and is hereby incorporated by reference.

### BACKGROUND

The human MAPT gene encodes 6 different isoforms of tau: 2N4R, 1N4R, 0N4R, 2N3R, 1N3R, 0N3R. The difference between transcripts containing the 4R (4 repeat) versus 3R (3 repeat) tau is based on the inclusion (4R) or exclusion (3R) of exon 10. Humans normally express equal ratios of 3R and 4R tau. In some tauopathies, such as Alzheimer's disease, experimental evidence from post-mortem brain tissue suggest that the insoluble aggregates of tau are comprised of 3R and 4R tau. In rarer tauopathy diseases, such as progressive supranuclear palsy (PSP) and cortical basal degeneration (CBD), the 4R tau proteins are the aggregate-prone species of tau. The reasons underlying these differential types of aggregates in diseases is unknown.

One major challenge, to date, has been developing assays to measure the mRNA of different isoforms of tau. For example, the “R/repeat” domain of tau makes it challenging to design primer pairs and TaqMan probes to measure 3R tau. Because of this paucity of assays for measuring certain isoforms of tau, it is challenging to accurately test the specificity of reagents (e.g., siRNAs) that should only reduce 4R tau and not affect 3R tau, versus reagents (e.g., siRNAs) that should reduce both.

### SUMMARY

Tau reporter compositions, tau reporter cells, and tau reporter animals are provided that comprise a four-repeat (4R) tau isoform linked to a first reporter protein and a three-repeat (3R) tau isoform linked to a second reporter protein that is different from the first reporter protein. Methods are provided for making such tau reporter cells and tau reporter animals and for using such tau reporter cells and tau reporter animals for assessing the activity of tau-targeting reagents.

In one aspect, provided is a cell comprising a four-repeat (4R) tau isoform linked to a first reporter protein and a three-repeat (3R) tau isoform linked to a second reporter protein that is different from the first reporter protein. In some such cells, the cell comprises a first fusion protein comprising the 4R tau isoform fused to the first reporter protein and a second fusion protein comprising the 3R tau isoform fused to the second reporter protein. In some such cells, the cell comprises a first nucleic acid encoding the first fusion protein and a second nucleic acid encoding the second fusion protein, wherein the cell expresses the first fusion protein and the second fusion protein.

In some such cells, the cell comprises a first nucleic acid comprising a coding sequence for the 4R tau isoform and a coding sequence for the first reporter protein and a second nucleic acid comprising a coding sequence for the 3R tau isoform and a coding sequence for the second reporter protein, wherein the cell expresses the 4R tau isoform, the 3R tau isoform, the first reporter protein, and the second reporter protein. In some such cells, the coding sequence for the 4R tau isoform and the coding sequence for the first reporter protein are separated by a coding sequence for a first 2A peptide, and the coding sequence for the 3R tau isoform and the coding sequence for the second reporter protein are separated by a coding sequence for a second 2A peptide. In some such cells, the first 2A peptide is a first P2A peptide, and the second 2A peptide is a second P2A peptide.

In some such cells, the first nucleic acid and the second nucleic acid are integrated into the genome of the cell. In some such cells, the cell comprises a viral vector comprising the first nucleic acid and the second nucleic acid. In some such cells, the viral vector is a lentivirus vector or an adeno-associated virus (AAV) vector. In some such cells, the cell comprises a first viral vector comprising the first nucleic acid and a second viral vector comprising the second nucleic acid. In some such cells, the first viral vector and the second viral vector are lentivirus vectors or adeno-associated virus (AAV) vectors.

In some such cells, the first reporter protein is a first fluorescent reporter protein, and the second reporter protein is a second fluorescent reporter protein. In some such cells, the first reporter protein is eYFP, and the second reporter protein is mCherry. In some such cells, the 4R tau isoform and the 3R tau isoform are human.

In some such cells, the 4R tau isoform is a 2N4R tau isoform. In some such cells, the 3R tau isoform is a 2N3R tau isoform. In some such cells, the 4R tau isoform is a 2N4R tau isoform, and wherein the 3R tau isoform is a 2N3R tau isoform. In some such cells, the 4R tau isoform comprises the sequence set forth in SEQ ID NO: 13, and the 3R tau isoform comprises the sequence set forth in SEQ ID NO: 14.

In some such cells, the 4R tau isoform is a 1N4R tau isoform. In some such cells, the 3R tau isoform is a 1N3R tau isoform. In some such cells, the 4R tau isoform is a 1N4R tau isoform, and wherein the 3R tau isoform is a 1N3R tau isoform. In some such cells, the 4R tau isoform comprises the sequence set forth in SEQ ID NO: 23, 27, 31, or 47, and the 3R tau isoform comprises the sequence set forth in SEQ ID NO: 24, 28, 32, or 49.

In some such cells, the cell is a mammalian cell. In some such cells, the cell is a human cell. In some such cells, the cell is an immortalized cell. In some such cells, the cell is a HEK293 cell.

In another aspect, provided are populations of cells comprising a plurality of any of the above cells.

In another aspect, provided is a non-human animal comprising a four-repeat (4R) tau isoform linked to a first reporter protein and a three-repeat (3R) tau isoform linked to a second reporter protein that is different from the first reporter protein. In some such non-human animals, the non-human animal comprises a first fusion protein comprising the 4R tau isoform fused to the first reporter protein and a second fusion protein comprising the 3R tau isoform fused to the second reporter protein. In some such non-human animals, the non-human animal comprises a first nucleic acid encoding the first fusion protein and a second nucleic acid encoding the second fusion protein, wherein the non-human animal expresses the first fusion protein and the second fusion protein.

In some such non-human animals, the non-human animal comprises a first nucleic acid comprising a coding sequence for the 4R tau isoform and a coding sequence for the first reporter protein and a second nucleic acid comprising a coding sequence for the 3R tau isoform and a coding sequence for the second reporter protein, wherein the non-human animal expresses the 4R tau isoform, the 3R tau isoform, the first reporter protein, and the second reporter protein. In some such non-human animals, the coding sequence for the 4R tau isoform and the coding sequence for the first reporter protein are separated by a coding sequence for a first 2A peptide, and the coding sequence for the 3R tau isoform and the coding sequence for the second reporter protein are separated by a coding sequence for a second 2A peptide. In some such non-human animals, the first 2A peptide is a first P2A peptide, and the second 2A peptide is a second P2A peptide.

In some such non-human animals, the first nucleic acid and the second nucleic acid are integrated into the genome of the non-human animal. In some such non-human animals, the non-human animal comprises a viral vector comprising the first nucleic acid and the second nucleic acid. In some such non-human animals, the viral vector is a lentivirus vector or an adeno-associated virus (AAV) vector. In some such non-human animals, the non-human animal comprises a first viral vector comprising the first nucleic acid and a second viral vector comprising the second nucleic acid. In some such non-human animals, the first viral vector and the second viral vector are lentivirus vectors or adeno-associated virus (AAV) vectors.

In some such non-human animals, the first reporter protein is a first fluorescent reporter protein, and the second reporter protein is a second fluorescent reporter protein. In some such non-human animals, the first reporter protein is eYFP, and the second reporter protein is mCherry.

In some such non-human animals, the 4R tau isoform and the 3R tau isoform are human, optionally wherein the 4R tau isoform and the 3R tau isoform each comprise a R5L mutation, a L237V mutation, or a G243V mutation, or optionally wherein the 4R tau isoform and the 3R tau isoform each comprise a N279K mutation, a L284R mutation, or a S285R mutation.

In some such non-human animals, the 4R tau isoform is a 2N4R tau isoform. In some such non-human animals, the 3R tau isoform is a 2N3R tau isoform. In some such non-human animals, the 4R tau isoform is a 2N4R tau isoform, and wherein the 3R tau isoform is a 2N3R tau isoform. In some such non-human animals, the 4R tau isoform comprises the sequence set forth in SEQ ID NO: 13, and the 3R tau isoform comprises the sequence set forth in SEQ ID NO: 14.

In some such non-human animals, the 4R tau isoform is a 1N4R tau isoform. In some such non-human animals, the 3R tau isoform is a 1N3R tau isoform. In some such non-human animals, the 4R tau isoform is a 1N4R tau isoform, and wherein the 3R tau isoform is a 1N3R tau isoform. In some such non-human animals, the 4R tau isoform comprises the sequence set forth in SEQ ID NO: 23, 27, 31, or 47, and the 3R tau isoform comprises the sequence set forth in SEQ ID NO: 24, 28, 32, or 49.

In some such non-human animals, the non-human animal is a mammal. In some such non-human animals, the non-human animal is a rodent. In some such non-human animals, the non-human animal is a mouse. In some such non-human animals, the non-human animal is a rat.

In some such non-human animals, the 4R tau isoform, the first reporter protein, the 3R tau isoform, and the second reporter protein are expressed in neurons of the central nervous system of the non-human animal. In some such non-human animals, the non-human animal comprises filamentous tau inclusions.

In another aspect, provided are methods of assessing the activity of a tau-targeting reagent. Some such methods comprise: (a) administering the tau-targeting reagent to any of the above cells; and (b) assessing the activity of the tau-targeting reagent in the cell. In some such methods, the activity of the tau-targeting reagent is assessed compared to a control cell that is not administered the tau-targeting reagent or is assessed compared to prior to administering the tau-targeting reagent.

In some such methods, the assessing comprises measuring one or more of 4R tau messenger RNA expression, first reporter protein messenger RNA expression, and second reporter protein messenger RNA expression. In some such methods, the assessing comprises measuring 4R tau isoform messenger RNA expression and second reporter protein messenger RNA expression, wherein a larger relative decrease in 4R tau isoform messenger RNA expression compared to second reporter protein messenger RNA expression after administering the tau-targeting reagent to the cell indicates that the tau-targeting reagent is a 4R-preferential tau targeting reagent, optionally wherein a decrease of at least 70% in 4R tau isoform messenger RNA expression and a decrease of no more than 30% in second reporter protein messenger RNA expression after administering the tau-targeting reagent to the cell indicates that the tau-targeting reagent is a 4R-preferential tau targeting reagent. In some such methods, the assessing comprises measuring first reporter protein messenger RNA expression and second reporter protein messenger RNA expression, wherein a larger relative decrease in first reporter protein messenger RNA expression compared to second reporter protein messenger RNA expression after administering the tau-targeting reagent to the cell indicates that the tau-targeting reagent is a 4R-preferential tau targeting reagent, optionally wherein a decrease of at least 70% in first reporter protein messenger RNA expression and a decrease of no more than 30% in second reporter protein messenger RNA expression after administering the tau-targeting reagent to the cell indicates that the tau-targeting reagent is a 4R-preferential tau targeting reagent.

In some such methods, the assessing comprises measuring one or more of first reporter protein expression and second reporter protein expression. In some such methods, the assessing comprises measuring first reporter protein expression and second reporter protein expression, wherein a larger relative decrease in first reporter protein expression compared to second reporter protein expression after administering the tau-targeting reagent to the cell indicates that the tau-targeting reagent is a 4R-preferential tau targeting reagent, optionally wherein a decrease of at least 70% in first reporter protein expression and a decrease of no more than 30% in second reporter protein expression after administering the tau-targeting reagent to the cell indicates that the tau-targeting reagent is a 4R-preferential tau targeting reagent.

In some such methods, the first reporter protein is a first fluorescent reporter protein, and the second reporter protein is a second fluorescent reporter protein, and the assessing in step (b) comprises immunofluorescence staining or flow cytometry. In some such methods, the assessing in step (b) comprises assessing tau hyperphosphorylation or tau aggregation.

In some such methods, the tau-targeting reagent is an RNAi agent or an antisense oligonucleotide. In some such methods, the tau-targeting reagent is an intrabody. In some such methods, the tau-targeting reagent is a nuclease agent. In some such methods, the nuclease agent comprises a Cas protein and a guide RNA designed to target a guide RNA target sequence in a tau coding sequence.

In another aspect, provided are methods of assessing the activity of a tau-targeting reagent in vivo. Some such methods comprise: (a) administering the tau-targeting reagent to any of the above non-human animals; and (b) assessing the activity of the tau-targeting reagent in the non-human animal. In some such methods, the activity of the tau-targeting reagent is assessed compared to a control non-human animal that is not administered the tau-targeting reagent or is assessed compared to prior to administering the tau-targeting reagent.

In some such methods, the assessing comprises measuring one or more of 4R tau messenger RNA expression, first reporter protein messenger RNA expression, and second reporter protein messenger RNA expression. In some such methods, the assessing comprises measuring 4R tau isoform messenger RNA expression and second reporter protein messenger RNA expression, wherein a larger relative decrease in 4R tau isoform messenger RNA expression compared to second reporter protein messenger RNA expression after administering the tau-targeting reagent to the non-human animal indicates that the tau-targeting reagent is a 4R-preferential tau targeting reagent, optionally wherein a decrease of at least 70% in 4R tau isoform messenger RNA expression and a decrease of no more than 30% in second reporter protein messenger RNA expression after administering the tau-targeting reagent to the non-human animal indicates that the tau-targeting reagent is a 4R-preferential tau targeting reagent. In some such methods, the assessing comprises measuring first reporter protein messenger RNA expression and second reporter protein messenger RNA expression, wherein a larger relative decrease in first reporter protein messenger RNA expression compared to second reporter protein messenger RNA expression after administering the tau-targeting reagent to the non-human animal indicates that the tau-targeting reagent is a 4R-preferential tau targeting reagent, optionally wherein a decrease of at least 70% in first reporter protein messenger RNA expression and a decrease of no more than 30% in second reporter protein messenger RNA expression after administering the tau-targeting reagent to the non-human animal indicates that the tau-targeting reagent is a 4R-preferential tau targeting reagent.

In some such methods, the assessing comprises measuring one or more of first reporter protein expression and second reporter protein expression. In some such methods, the assessing comprises measuring first reporter protein expression and second reporter protein expression, wherein a larger relative decrease in first reporter protein expression compared to second reporter protein expression after administering the tau-targeting reagent to the non-human animal indicates that the tau-targeting reagent is a 4R-preferential tau targeting reagent, optionally wherein a decrease of at least 70% in first reporter protein expression and a decrease of no more than 30% in second reporter protein expression after administering the tau-targeting reagent to the non-human animal indicates that the tau-targeting reagent is a 4R-preferential tau targeting reagent.

In some such methods, the first reporter protein is a first fluorescent reporter protein, and the second reporter protein is a second fluorescent reporter protein, and the assessing in step (b) comprises immunofluorescence staining or flow cytometry. In some such methods, the assessing in step (b) comprises assessing tau hyperphosphorylation or tau aggregation.

In some such methods, the tau-targeting reagent is an RNAi agent or an antisense oligonucleotide. In some such methods, the tau-targeting reagent is an intrabody. In some such methods, the tau-targeting reagent is a nuclease agent. In some such methods, the nuclease agent comprises a Cas protein and a guide RNA designed to target a guide RNA target sequence in a tau coding sequence.

In some such methods, the assessing is in neurons in the central nervous system of the non-human animal.

In another aspect, provided are compositions. Some such compositions comprise: (a) a four-repeat (4R) tau isoform linked to a first reporter protein and a three-repeat (3R) tau isoform linked to a second reporter protein that is different from the first reporter protein; or (b) a first nucleic acid encoding the 4R tau isoform linked to the first reporter protein and a second nucleic acid encoding the 3R tau isoform linked to the second reporter protein.

In some such compositions, the composition comprises a first fusion protein comprising the 4R tau isoform fused to the first reporter protein and a second fusion protein comprising the 3R tau isoform fused to the second reporter protein, or wherein the first nucleic acid encodes the first fusion protein and the second nucleic acid encodes the second fusion protein. In some such compositions, the first nucleic acid comprises a coding sequence for the 4R tau isoform and a coding sequence for the first reporter protein separated by a coding sequence for a first 2A peptide, and wherein the second nucleic acid comprises a coding sequence for the 3R tau isoform and a coding sequence for the second reporter protein separated by a coding sequence for a second 2A peptide. In some such compositions, the first 2A peptide is a first P2A peptide, and the second 2A peptide is a second P2A peptide.

In some such compositions, the first nucleic acid and the second nucleic acid are in a viral vector. In some such compositions, the viral vector is a lentivirus vector or an adeno-associated virus (AAV) vector. In some such compositions, the first nucleic acid is in a first viral vector, and the second nucleic acid is in a second viral vector. In some such compositions, the first viral vector and the second viral vector are lentivirus vectors or adeno-associated virus (AAV) vectors.

In some such compositions, the first reporter protein is a first fluorescent reporter protein, and the second reporter protein is a second fluorescent reporter protein. In some such compositions, the first reporter protein is eYFP, and the second reporter protein is mCherry.

In some such compositions, the 4R tau isoform and the 3R tau isoform are human.

In some such compositions, the 4R tau isoform is a 2N4R tau isoform. In some such compositions, the 3R tau isoform is a 2N3R tau isoform. In some such compositions, the 4R tau isoform is a 2N4R tau isoform, and wherein the 3R tau isoform is a 2N3R tau isoform. In some such compositions, the 4R tau isoform comprises the sequence set forth in SEQ ID NO: 13, and the 3R tau isoform comprises the sequence set forth in SEQ ID NO: 14.

In some such compositions, the 4R tau isoform is a 1N4R tau isoform. In some such compositions, the 3R tau isoform is a 1N3R tau isoform. In some such compositions, the 4R tau isoform is a 1N4R tau isoform, and wherein the 3R tau isoform is a 1N3R tau isoform. In some such compositions, the 4R tau isoform comprises the sequence set forth in SEQ ID NO: 23, 27, 31, or 47, and the 3R tau isoform comprises the sequence set forth in SEQ ID NO: 24, 28, 32, or 49.

In another aspect, provided is a cell comprising any of the above compositions. In another aspect, provided is a non-human animal comprising any of the above compositions.

In another aspect, provided is a method of making any of the above cells. Some such methods comprise introducing into the cell the four-repeat (4R) tau isoform linked to the first reporter protein and the three-repeat (3R) tau isoform linked to the second reporter protein, or introducing into the cell a first nucleic acid encoding the 4R tau isoform linked to the first reporter protein and a second nucleic acid encoding the 3R tau isoform linked to the second reporter protein.

In another aspect, provided is a method of making any of the above non-human animals. Some such methods comprise administering to the non-human animal the four-repeat (4R) tau isoform linked to the first reporter protein and the three-repeat (3R) tau isoform linked to the second reporter protein, or administering to the non-human animal a first nucleic acid encoding the 4R tau isoform linked to the first reporter protein and a second nucleic acid encoding the 3R tau isoform linked to the second reporter protein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows different four-repeat (4R) and three-repeat (3R) tau isoforms. Exon 10 encodes the “R2 domain,” which distinguishes 4R tau from 3R tau.

FIG. 2 shows relative expression of 2N4R-eYFP and 2N3R-mCherry mRNA in dual reporter 2N4R-eYFP/2N3R-mCherry cell lines using eYFP-specific and mCherry-specific TaqMan probes.

FIG. 3 shows immunofluorescence images from 2N4R-eYFP/2N3R-mCherry cell line clones C2, C3, and C5. The top row shows eYFP, the middle row shows mCherry, and the third row shows merged eYFP and mCherry images.

FIG. 4 shows immunofluorescence images from an untreated 2N4R-eYFP/2N3R-mCherry cell line (first column) or the cell line treated with 10 nM total tau siRNA (targeting both 3R and 4R tau) or 10 nM mCherry siRNA (HAIJE-000013).

FIG. 5 shows immunofluorescence images from an untreated 2N4R-eYFP/2N3R-mCherry cell line (first column) or the cell line treated with 10 nM total YFP siRNA (P-002048-01) or 10 nM mCherry siRNA (HAIJE-000015 or HAIJE-000017).

FIG. 6 shows possible criteria for selecting “4R-specific” siRNAs and “4R-preferential siRNAs.”

FIG. 7 shows percent RNA knockdown as measured by 4R tau and mCherry TaqMan assays for the 65 candidate 4R tau siRNAs.

FIG. 8 shows the correlation between percent 4R tau knockdown and percent eYFP knockdown as measured by TaqMan.

FIG. 9A TaqMan qPCR data of expression levels of 4R tau and mCherry (3R tau surrogate) following treatment of HEK293-2N4R-YFP+2N3R-mCherry dual reporter cells with 1 nM of 65 different GalNAc-conjugated siRNAs that were produced by walking the entirety of exon 10 of the MAPT gene. Lipofectamine was used as a negative control. A total tau siRNA was also used as a control.

FIG. 9B shows relative 4R tau and mCherry expression for thirteen 4R-preferential siRNAs (1 nM and 0.1 nM). Lipofectamine was used as a negative control. Two total tau siRNAs were also used as controls.

FIG. 10A shows IC50 graphs for two candidate 4R siRNAs using the 3R/4R-dual reporter. The siRNAs were tested at eight different concentrations. FIG. 10B shows IC50 graphs for two candidate 4R siRNAs using a 3R-mCherry reporter. The siRNAs were tested at eight different concentrations. FIG. 10C shows IC50 graphs for two candidate 4R siRNAs using a 4R-YFP reporter. The siRNAs were tested at eight different concentrations.

FIG. 11A shows IC50 graphs for two candidate 4R siRNAs using the 3R/4R-dual reporter. The siRNAs were tested at eight different concentrations. FIG. 11B shows IC50 graphs for two candidate 4R siRNAs using a 3R-mCherry reporter. The siRNAs were tested at eight different concentrations. FIG. 11C shows IC50 graphs for two candidate 4R siRNAs using a 4R-YFP reporter. The siRNAs were tested at eight different concentrations.

FIG. 12 shows validation of four 4R-preferential siRNAs in 4R tau protein knockdown using dot blotting with 4R-specific and 3R-specific antibodies. Validation of the antibodies with recombinant tau 1N3R or 1N4R proteins to confirm specificity is shown on the left, and testing with lysates from siRNA-treated dual-reporter cells is shown on the right. The experimental set-up is shown at the top.

FIG. 13 shows validation of four 4R-preferential siRNAs in 4R tau protein knockdown using total tau and 3R tau ELISAs. In the left panel, a commercially available total tau ALPHALISA (Perkin Elmer, cat no: AL271C) was used to measure total soluble tau according to the manufacturer's instructions. In the right panel, 4R tau protein was measured using the PathScan ELISA kit from Cell Signaling Technologies (cat no: 29443). The experimental set-up is shown at the top.

FIG. 14 shows RT-qPCR data of expression levels of total tau and 4R tau on RNA extracted from the brains of humanized MAPT mice treated with 4R tau siRNAs. Values are graphed as relative expression (1/2ΔΔct) to the naïve group and normalized to mouse GAPDH.

FIG. 15 shows schematics for 1N4R tau(R5L)-P2A-eYFP and 1N3R tau(R5L)-P2A-mCherry, 1N4R tau(L237V)-P2A-eYFP and 1N3R tau(L237V)-P2A-mCherry, and 1N4R tau(G243V)-P2A-eYFP and 1N3R tau(G243V)-P2A-mCherry for use in creating a mouse model for in vivo testing of 4R tau targeting reagents.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” (amino-terminus) and a “C-terminus” (carboxy-terminus or carboxyl-terminus). The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “targeting vector” refers to a recombinant nucleic acid that can be introduced by homologous recombination, non-homologous-end-joining-mediated ligation, or any other means of recombination to a target position in the genome of a cell.

The term “isolated” with respect to cells, tissues, proteins, and nucleic acids includes cells, tissues, proteins, and nucleic acids that are relatively purified with respect to other bacterial, viral, cellular, or other components that may normally be present in situ, up to and including a substantially pure preparation of the cells, tissues, proteins, and nucleic acids. The term “isolated” also includes cells, tissues, proteins, and nucleic acids that have no naturally occurring counterpart, have been chemically synthesized and are thus substantially uncontaminated by other cells, tissues, proteins, and nucleic acids, or has been separated or purified from most other components (e.g., cellular components or organism components) with which they are naturally accompanied (e.g., other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a rat cell or rat. For example, an endogenous MAPT sequence of a human cell refers to a native MAPT sequence that naturally occurs at the MAPT locus in the human cell.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a tau protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “MAPT locus” may refer to the specific location of a MAPT gene, MAPT DNA sequence, tau-encoding sequence, or MAPT position on a chromosome of the genome of an organism that has been identified as to where such a sequence resides. A “MAPT locus” may comprise a regulatory element of a MAPT gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

The “coding region” or “coding sequence” of a gene consists of the portion of a gene's DNA or RNA, composed of exons, that codes for a protein. The region begins at the start codon on the 5′ end and ends at the stop codon on the 3′ end.

A “promoter” is a regulatory region of DNA usually comprising a TATA box capable of directing RNA polymerase II to initiate RNA synthesis at the appropriate transcription initiation site for a particular polynucleotide sequence. In some cases, a promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., an organism or body or a cell or tissue within an organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values±5% of a stated value.

The term “and/or” refers to and encompasses any and all possible combinations of one or more of the associated listed items, as well as the lack of combinations when interpreted in the alternative (“or”).

The term “or” refers to any one member of a particular list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Tau reporter compositions, tau reporter cells, and tau reporter animals are provided that comprise a four-repeat (4R) tau isoform linked to a first reporter protein and a three-repeat (3R) tau isoform linked to a second reporter protein that is different from the first reporter protein. Methods are provided for making such tau reporter cells and tau reporter animals and for using such tau reporter cells and tau reporter animals for assessing the activity of tau-targeting reagents.

The human MAPT gene encodes 6 different isoforms of tau: 2N4R, 1N4R, 0N4R, 2N3R, 1N3R, 0N3R. The difference between transcripts containing the 4R (4 repeat) versus 3R (3 repeat) tau is based on the inclusion (4R) or exclusion (3R) of exon 10. Humans normally express equal ratios of 3R and 4R tau. In some tauopathies, such as Alzheimer's disease, experimental evidence from post-mortem brain tissue suggest that the insoluble aggregates of tau are comprised of 3R and 4R tau. In rarer tauopathy diseases, such as progressive supranuclear palsy (PSP), and cortical basal degeneration (CBD), the 4R tau proteins are the aggregate-prone species of tau. The reasons underlying these differential types of aggregates in diseases is unknown. Therapies that target total tau (e.g., 3R+4R tau) versus only 4R tau could have beneficial effects in different disease states.

One major challenge, to date, has been developing assays to measure the mRNA of different isoforms of tau. For example, the “R/repeat” domain of tau makes it challenging to design primer pairs and TaqMan probes to measure 3R tau. Because of this paucity of assays for measuring certain isoforms of tau, it is challenging to accurately test the specificity of reagents (e.g., siRNAs) that should only reduce 4R tau and not affect 3R tau, versus reagents (e.g., siRNAs) that should reduce both (e.g., total tau targeting strategy).

The tau reporters, tau reporter cells, and tau reporter non-human animals are able to identify compounds (e.g., siRNA or gRNA) which target total tau versus compounds that specifically target 4R tau. Until now, screening of 4R-tau-specific reagents such as siRNAs was impossible due to the lack of protein and mRNA assays to measure 3R tau specifically.

### II. Compositions Comprising Tau 3R and Tau 4R Reporters

Provided herein are compositions comprising tau 3R and tau 4R reporters that can be used in assays to distinguish tau-targeting reagent targeting 4R tau specifically or selectively versus reagents targeting both 4R tau and 3R tau.

Tau is an intracellular, microtubule-associated protein that binds to and stabilizes microtubules. It is predominantly expressed in neurons. Tau has a role in stabilizing neuronal microtubules and thus in promoting axonal outgrowth. In Alzheimer's disease (AD) and a family of related neurodegenerative diseases called tauopathies, tau protein is abnormally hyperphosphorylated and aggregated into bundles of filaments (paired helical filaments), which manifest as neurofibrillary tangles. Tauopathies are a group of heterogeneous neurodegenerative conditions characterized by deposition of abnormal tau in the brain. Tau tangle burden positively correlates with cognitive decline and dying neurons. In Alzheimer's disease (AD), extracellular amyloid beta triggers aggregation of both 3R and 4R isoforms of tau (secondary tauopathy), leading to cognitive decline. In primary tauopathies, tau isoforms differentially aggregate due to unknown reasons/triggers. For example, 4R aggregates are associated with progressive supranuclear palsy (PSP) and corticobasal degeneration (CBD), whereas 3R aggregates are associated with Pick's disease.

Tau is encoded by the MAPT gene, which comprises 16 exons. Exons 0 and 1 encode the 5′ untranslated region (UTR) of the MAPT mRNA, while exon 14 encodes part of the 3′ UTR. Exons 4a, 6, and 8 are only transcribed in peripheral tissues. Exons 2 and 3 encode 29-amino acid residue inserts near the amino-terminus (N1 and N2), and exons 9-12 encode microtubule binding domain repeats near the carboxyl-terminus (R1-R4). Alternative splicing of MAPT results in six isoforms ranging from 352 to 441 amino acids. Tau isoforms vary and are named based on the number of amino-terminal inserts (0N, 1N, and 2N) and the number of carboxyl-terminal microtubule binding domain repeats (3R and 4R). Depending on splicing, the repeat domain of the tau protein has either three or four repeat regions that constitute the aggregation-prone core of the protein, which is often termed the repeat domain (RD). The three four-repeat (4R) tau isoforms are 2N4R, 1N4R, and 0N4R. The three three-repeat (3R) tau isoforms are 2N3R, 1N3R, and 0N3R.

A composition described herein can comprise a 4R tau isoform linked to a first reporter protein and a 3R tau isoform linked to a second reporter protein that is different from the first reporter protein. Alternatively, a composition described herein can comprise a first nucleic acid encoding a 4R tau isoform linked to a first reporter protein and a second nucleic acid encoding a 3R tau isoform linked to a second reporter protein. By “linked” it is meant that the tau isoform and the linked reporter protein are part of the same protein (i.e., a fusion protein) or are expressed from the same messenger RNA. In a fusion protein, the tau isoform and reporter protein can be directly fused to each other or can fused to each other via a linker. In a specific example, the tau isoform and the linked reporter protein are fused to each other via a linker. In a first example, the composition can comprise a first fusion protein comprising a 4R tau isoform fused to a first reporter protein and a second fusion protein comprising a 3R tau isoform fused to a second reporter protein that is different from the first reporter protein. For example, the composition can comprise a first nucleic acid (e.g., expression cassette) encoding the first fusion protein and a second nucleic acid (e.g., expression cassette) encoding the second fusion protein. In a second example, the composition can comprise a first nucleic acid (e.g., expression cassette) comprising a coding sequence for a 4R tau isoform and a coding sequence for a first reporter protein and a second nucleic acid (e.g., expression cassette) comprising a coding sequence for a 3R tau isoform and a coding sequence for a second reporter protein that is different from the first reporter protein, wherein the coding sequence for the 4R tau isoform and the coding sequence for the first reporter protein are separated by the coding sequence for a first 2A peptide, and the coding sequence for the 3R tau isoform and the coding sequence for the second reporter protein are separated by the coding sequence for a second 2A peptide.

2A peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4):e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin. Biol. Ther. 5(5):627-638, herein incorporated by reference in its entirety for all purposes. Examples of 2A peptides that can be used include Thoseaasigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 9); P2A (ATNFSLLKQAGDVEENPGP; SEQ ID NO: 10); E2A (QCTNYALLKLAGDVESNPGP; SEQ ID NO: 11); and F2A (VKQTLNFDLLKLAGDVESNPGP; SEQ ID NO: 12). GSG residues can be added to the 5′ end of any of these peptides to improve cleavage efficiency. In a specific example, P2A is used.

In a composition comprising the first nucleic acid and the second nucleic acid, such nucleic acids can be RNA (e.g., messenger RNA (mRNA)) or DNA, can be single-stranded or double-stranded, and can be linear or circular. DNA can be part of a vector, such as an expression vector or a targeting vector. The vector can also be a viral vector such as adenoviral, adeno-associated viral, lentiviral, and retroviral vectors.

Optionally, the nucleic acids can be codon-optimized for efficient translation into protein in a particular cell or organism. For example, the nucleic acid can be modified to substitute codons having a higher frequency of usage in a human cell, a non-human cell, a mammalian cell, a rodent cell, a mouse cell, a rat cell, a bacterial cell, a yeast cell, or any other host cell of interest, as compared to the naturally occurring polynucleotide sequence.

The compositions described herein can be in vitro, they can be within a cell (e.g., an embryonic stem cell), or they can be in a non-human animal. The cell can be in any type of cell from any organism, such as an embryonic stem cell (e.g., a mouse or a rat embryonic stem cell) or an induced pluripotent stem cell (e.g., a human induced pluripotent stem cell). The non-human animal can be any type of suitable non-human animal as described further elsewhere herein.

The nucleic acids or expression cassettes can be stably integrated into the genome (i.e., into a chromosome) of a cell or non-human animal or they can be located outside of a chromosome (e.g., extrachromosomally replicating DNA). Stably integrated expression cassettes or nucleic acids can be randomly integrated into the genome of the cell or non-human animal (i.e., transgenic), or they can be integrated into a predetermined region of the genome of the cell or non-human animal (i.e., knock-in). In one example, a nucleic acid or expression cassette is stably integrated into a safe harbor locus as described elsewhere herein. The target genomic locus at which a nucleic acid or expression cassette is stably integrated can be heterozygous for the nucleic acid or expression cassette or homozygous for the nucleic acid or expression cassette.

A nucleic acid or expression cassette described herein can be operably linked to any suitable promoter for expression in vivo within a non-human animal or in vitro or ex vivo within a cell. The non-human animal can be any suitable non-human animal as described elsewhere herein. As one example, a nucleic acid or expression cassette can be operably linked to an endogenous promoter at a target genomic locus, such as a Rosa26 promoter. Alternatively, a nucleic acid or expression cassette can be operably linked to an exogenous promoter, such as a constitutively active promoter (e.g., a CAG promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, or a zygote. Such promoters can be, for example, conditional promoters, inducible promoters, constitutive promoters, or tissue-specific promoters.

The nucleic acids and expression cassettes described herein can be in any form. For example, an expression cassette can be in a vector or plasmid, such as a viral vector. The expression cassette can be operably linked to a promoter in an expression construct capable of directing expression of a protein or RNA. Alternatively, an expression cassette can be in a targeting vector. For example, the targeting vector can comprise homology arms flanking the expression cassette, wherein the homology arms are suitable for directing recombination with a desired target genomic locus to facilitate genomic integration and/or replacement of endogenous sequence.

When a composition described herein is in a cell or non-human animal, the composition can comprise a first nucleic acid (e.g., expression cassette) encoding a first fusion protein and a second nucleic acid (e.g., expression cassette) encoding a second fusion protein, and the first fusion protein and the second fusion protein can be stably expressed in the cell or non-human animal. For example, the first nucleic acid (e.g., expression cassette) and the second nucleic acid (e.g., expression cassette) can be genomically integrated in the cell or non-human animal and operably linked to promoters active in the cell or non-human animal. For example, the first nucleic acid and the second nucleic acid can be genomically integrated in the germline of the non-human animal. Likewise, when a composition described herein is in a cell or non-human animal, the composition can comprise a first nucleic acid (e.g., expression cassette) comprising a coding sequence for a 4R tau isoform and a coding sequence for a first reporter protein (e.g., separated by a first 2A coding sequence) and a second nucleic acid (e.g., expression cassette) comprising a coding sequence for a 3R tau isoform and a coding sequence for a second reporter protein (e.g., separated by a second 2A coding sequence), and the 4R tau isoform, the first reporter protein, the 3R tau isoform, and the second reporter protein can be stably expressed in the cell or non-human animal. For example, the first nucleic acid (e.g., expression cassette) and the second nucleic acid (e.g., expression cassette) can be genomically integrated in the cell or non-human animal and operably linked to promoters active in the cell or non-human animal. For example, the first nucleic acid and the second nucleic acid can be genomically integrated in the germline of the non-human animal. The nucleic acids (e.g., expression cassettes) can be randomly integrated in the genome of the cell or non-human animal, or they can be integrated into a target genomic locus, such as a safe harbor locus. Any target genomic locus capable of expressing a gene can be used. The promoter can be any suitable promoter. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter).

An example of a target genomic locus into which the nucleic acids or expression cassettes described herein can be stably integrated is a safe harbor locus in the genome of the cell or non-human animal. Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

For example, the Rosa26 locus and its equivalent in humans offer an open chromatin configuration in all tissues and is ubiquitously expressed during embryonic development and in adults. See, e.g., Zambrowicz et al. (1997) Proc. Natl. Acad. Sci. USA 94:3789-3794, herein incorporated by reference in its entirety for all purposes. In addition, the Rosa26 locus can be targeted with high efficiency, and disruption of the Rosa26 gene produces no overt phenotype. Other examples of safe harbor loci include CCR5, HPRT, AAVS1, and albumin. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Biallelic targeting of safe harbor loci such as the Rosa26 locus has no negative consequences, so different genes or reporters can be targeted to the two Rosa26 alleles.

Nucleic acids (e.g., expression cassettes) integrated into a target genomic locus can be operably linked to an endogenous promoter at the target genomic locus or can be operably linked to an exogenous promoter that is heterologous to the target genomic locus.

The compositions described herein can comprise a vector (e.g., a viral vector) comprising the first nucleic acid (e.g., expression cassette) and the second nucleic acid (e.g., expression cassette), or a first vector (e.g., a viral vector) comprising the first nucleic acid (e.g., expression cassette) and a second vector (e.g., a viral vector) comprising the second nucleic acid (e.g., expression cassette).

A vector can comprise additional sequences such as, for example, replication origins, promoters, and genes encoding antibiotic resistance. Some vectors may be circular. Alternatively, the vector may be linear. Non-limiting exemplary vectors include plasmids, phagemids, cosmids, artificial chromosomes, minichromosomes, transposons, viral vectors, and expression vectors. The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression.

In one example, the nucleic acid or expression construct is in an AAV vector. The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

The 4R and 3R tau isoforms in the compositions can be from any animal or mammal, such as human, mouse, or rat. In a specific example, the 4R and 3R tau isoforms are human. The 4R and 3R tau isoforms can also be any combination of 4R and 3R tau isoforms, respectively. In one example, the 4R tau isoform is a 2N4R isoform. For example, the 4R tau isoform can comprise the sequence set forth in SEQ ID NO: 13, optionally wherein the 4R tau isoform is encoded by the sequence set forth in SEQ ID NO: 17. For example, in compositions in which the 4R tau isoform is fused to the first reporter protein, the fusion protein can comprise the sequence set forth in SEQ ID NO: 15, optionally wherein the fusion protein is encoded by the sequence set forth in SEQ ID NO: 19. In another example, the 4R tau isoform is a 1N4R isoform. For example, the 4R tau isoform can comprise the sequence set forth in SEQ ID NO: 23, 27, 31, or 47, optionally wherein the 4R tau isoform is encoded by the sequence set forth in SEQ ID NO: 25, 29, 33, or 48, respectively. For example, in compositions in which the 4R tau isoform is fused to the first reporter protein, the fusion protein can comprise the sequence set forth in SEQ ID NO: 35, optionally wherein the fusion protein is encoded by the sequence set forth in SEQ ID NO: 36. For example, in compositions in which the 4R tau isoform is fused to the first reporter protein, the fusion protein can comprise the sequence set forth in SEQ ID NO: 37, optionally wherein the fusion protein is encoded by the sequence set forth in SEQ ID NO: 38. For example, in compositions in which the 4R tau isoform is fused to the first reporter protein, the fusion protein can comprise the sequence set forth in SEQ ID NO: 39, optionally wherein the fusion protein is encoded by the sequence set forth in SEQ ID NO: 40.

In one example, the 3R tau isoform is a 2N3R isoform. For example, the 3R tau isoform can comprise the sequence set forth in SEQ ID NO: 14, optionally wherein the 3R tau isoform is encoded by the sequence set forth in SEQ ID NO: 18. For example, in compositions in which the 3R tau isoform is fused to the second reporter protein, the fusion protein can comprise the sequence set forth in SEQ ID NO: 16, optionally wherein the fusion protein is encoded by the sequence set forth in SEQ ID NO: 20. In another example, the 4R tau isoform is a 1N3R isoform. For example, the 3R tau isoform can comprise the sequence set forth in SEQ ID NO: 24, 28, 32, or 49, optionally wherein the 3R tau isoform is encoded by the sequence set forth in SEQ ID NO: 26, 30, 34, or 50, respectively. For example, in compositions in which the 3R tau isoform is fused to the second reporter protein, the fusion protein can comprise the sequence set forth in SEQ ID NO: 41, optionally wherein the fusion protein is encoded by the sequence set forth in SEQ ID NO: 42. For example, in compositions in which the 3R tau isoform is fused to the second reporter protein, the fusion protein can comprise the sequence set forth in SEQ ID NO: 43, optionally wherein the fusion protein is encoded by the sequence set forth in SEQ ID NO: 44. For example, in compositions in which the 3R tau isoform is fused to the second reporter protein, the fusion protein can comprise the sequence set forth in SEQ ID NO: 45, optionally wherein the fusion protein is encoded by the sequence set forth in SEQ ID NO: 46.

In one specific example, the 4R tau isoform is a 2N4R isoform, and the 3R tau isoform is a 2N3R isoform. For example, the 4R tau isoform can comprise the sequence set forth in SEQ ID NO: 13, and the 3R tau isoform can comprise the sequence set forth in SEQ ID NO: 14 (optionally encoded by SEQ ID NOS: 17 and 18, respectively). For example, the 4R tau isoform can be fused to the first reporter protein, and the fusion protein can comprise the sequence set forth in SEQ ID NO: 15, and the 3R tau isoform can be fused to the second reporter protein, and the fusion protein can comprise the sequence set forth in SEQ ID NO: 16 (optionally encoded by SEQ ID NOS: 19 and 20, respectively). In another specific example, the 4R tau isoform is a 1N4R isoform, and the 3R tau isoform is a 1N3R isoform. For example, the 4R tau isoform can comprise the sequence set forth in SEQ ID NO: 23, and the 3R tau isoform can comprise the sequence set forth in SEQ ID NO: 24 (optionally encoded by SEQ ID NOS: 25 and 26, respectively). For example, the 4R tau isoform can comprise the sequence set forth in SEQ ID NO: 27, and the 3R tau isoform can comprise the sequence set forth in SEQ ID NO: 28 (optionally encoded by SEQ ID NOS: 29 and 30, respectively). For example, the 4R tau isoform can comprise the sequence set forth in SEQ ID NO: 31, and the 3R tau isoform can comprise the sequence set forth in SEQ ID NO: 32 (optionally encoded by SEQ ID NOS: 33 and 34, respectively). For example, the 4R tau isoform can comprise the sequence set forth in SEQ ID NO: 47, and the 3R tau isoform can comprise the sequence set forth in SEQ ID NO: 49 (optionally encoded by SEQ ID NOS: 48 and 50, respectively). For example, the 4R tau isoform can be fused to the first reporter protein, and the fusion protein can comprise the sequence set forth in SEQ ID NO: 35, and the 3R tau isoform can be fused to the second reporter protein, and the fusion protein can comprise the sequence set forth in SEQ ID NO: 41 (optionally encoded by SEQ ID NOS: 36 and 42, respectively). For example, the 4R tau isoform can be fused to the first reporter protein, and the fusion protein can comprise the sequence set forth in SEQ ID NO: 37, and the 3R tau isoform can be fused to the second reporter protein, and the fusion protein can comprise the sequence set forth in SEQ ID NO: 43 (optionally encoded by SEQ ID NOS: 38 and 44, respectively). For example, the 4R tau isoform can be fused to the first reporter protein, and the fusion protein can comprise the sequence set forth in SEQ ID NO: 39, and the 3R tau isoform can be fused to the second reporter protein, and the fusion protein can comprise the sequence set forth in SEQ ID NO: 45 (optionally encoded by SEQ ID NOS: 40 and 46, respectively). In addition to the 2N4R/2N3R and 1N4R/1N3R combinations disclosed above, any other combination can be used (e.g., 0N4R/0N3R, 0N4R/1N3R, 0N4R/2N3R, 1N4R/0N3R, 1N4R/1N3R, 1N4R/2N3R, 2N4R/0N3R, 2N4R/1N3R, or 2N4R/2N3R).

The 4R and/or 3R tau isoforms can be wild type. Alternatively, the 4R and/or 3R tau isoforms can comprise a tau pathogenic mutation, such as a pro-aggregation mutation. Such a mutation can be, for example, a mutation that is associated with (e.g., segregates with) or causes a tauopathy. As one example, the mutation can be an aggregation-sensitizing mutation that sensitizes tau to seeding but does not result in tau readily aggregating on its own, or it can be a mutation that is known to cause aggregation of both 3R and 4R tau. For example, the tau mutation can be R5L, L237V, or G243V. R5L is a mutation reported in a PSP patient (although it is present in all six isoforms of tau, in the human carrier selective aggregation of only 4R tau is observed), and G243V and L237V are mutations that have been reported to selectively aggravate 4R aggregation. By R5L mutation is meant the human tau R5L mutation or a corresponding mutation in another tau protein when optimally aligned with the human tau protein. By L237V mutation is meant the human tau L237V mutation or a corresponding mutation in another tau protein when optimally aligned with the human tau protein. By G243V mutation is meant the human tau G243V mutation or a corresponding mutation in another tau protein when optimally aligned with the human tau protein. In one example, both the 4R and 3R tau isoforms comprise a R5L mutation. In one example, both the 4R and 3R tau isoforms comprise a L237V mutation. In one example, both the 4R and 3R tau isoforms comprise a G243V mutation. In another example, the tau mutation can be N279K, L284R, or S285R. These are all exon 10 mutants that have been reported in humans with PSP-like symptoms and pathology. By N279K mutation is meant the human tau N279K mutation or a corresponding mutation in another tau protein when optimally aligned with the human tau protein. By L284R mutation is meant the human tau L284R mutation or a corresponding mutation in another tau protein when optimally aligned with the human tau protein. By S285R mutation is meant the human tau S285R mutation or a corresponding mutation in another tau protein when optimally aligned with the human tau protein. In one example, both the 4R and 3R tau isoforms comprise a N279K mutation. In one example, both the 4R and 3R tau isoforms comprise a L284R mutation. In one example, both the 4R and 3R tau isoforms comprise a S285R mutation.

The first and second reporter proteins can be any suitable reporter proteins encoded by any suitable reporter gene. The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene. In a specific example, the first and second reporter proteins are fluorescent reporter proteins.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods. In a specific example, the first and second reporter proteins are a yellow fluorescent protein (e.g., eYFP) and a red fluorescent protein (e.g., mCherry), respectively, or vice versa.

Also provided herein is a cell or a population of cells comprising the compositions described herein. The cells can be any type of cell and can be in vitro, ex vivo, or in vivo. A cell or population of cells can be a monoclonal cell line or population of cells. The cell can be from any source. For example, the cell can be a eukaryotic cell, an animal cell, a plant cell, or a fungal (e.g., yeast) cell. Such cells can be fish cells or bird cells, or such cells can be mammalian cells, such as human cells, non-human mammalian cells, rodent cells, mouse cells, or rat cells. Mammals include, for example, humans, non-human primates, monkeys, apes, cats dogs, horses, bulls, deer, bison, sheep, rodents (e.g., mice, rats, hamsters, guinea pigs), livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. In a specific example, the cells are human cells (e.g., HEK293 cells).

The cell can be, for example, a totipotent cell or a pluripotent cell (e.g., an embryonic stem (ES) cell such as a rodent ES cell, a mouse ES cell, or a rat ES cell). Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cell can also be a primary somatic cell, or a cell that is not a primary somatic cell. Somatic cells can include any cell that is not a gamete, germ cell, gametocyte, or undifferentiated stem cell. The cell can also be a primary cell. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, somatic cells, hematopoietic cells, endothelial cells, epithelial cells, fibroblasts, mesenchymal cells, keratinocytes, melanocytes, monocytes, mononuclear cells, adipocytes, preadipocytes, neurons, glial cells, hepatocytes, skeletal myoblasts, and smooth muscle cells. For example, primary cells can be derived from connective tissues, muscle tissues, nervous system tissues, or epithelial tissues.

Such cells also include would normally not proliferate indefinitely but, due to mutation or alteration, have evaded normal cellular senescence and instead can keep undergoing division. Such mutations or alterations can occur naturally or be intentionally induced. Examples of immortalized cells include Chinese hamster ovary (CHO) cells, human embryonic kidney cells (e.g., HEK293 cells), and mouse embryonic fibroblast cells (e.g., 3T3 cells). Numerous types of immortalized cells are well known. Immortalized or primary cells include cells that are typically used for culturing or for expressing recombinant genes or proteins.

The cell can also be a differentiated cell, such as a neuronal cell (e.g., a human neuronal cell). The cell can be an isolated cell.

Also provided herein are non-human animals comprising the compositions described herein. Non-human animals can be any suitable non-human animal. The term “animal” includes any member of the animal kingdom, including, for example, mammals, fishes, reptiles, amphibians, birds, and worms. In a specific example, the non-human animal is a non-human mammal. Non-human mammals include, for example, non-human primates and rodents (e.g., mice and rats). The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Sv1m), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal wN, C57BL/6, C57BL/6J, C57BL/6ByJ, C57BL/6NJ, C57BL/10, C57BL/10ScSn, C57BL/10Cr, and C57BL/01a. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

### III. Tau Reporter Cells and Tau Reporter Non-Human Animals

Provided herein is a tau reporter cell or a population of tau reporter cells that can be used in cellular screening assays to distinguish tau-targeting reagent targeting 4R tau specifically or selectively versus reagents targeting both 4R tau and 3R tau. Likewise, provided herein are tau reporter non-human animals that can be used in in vivo screening assays to distinguish tau-targeting reagent targeting 4R tau specifically or selectively versus reagents targeting both 4R tau and 3R tau.

A tau reporter cell or tau reporter non-human animal can comprise a 4R tau isoform linked to a first reporter protein and a 3R tau isoform linked to a second reporter protein that is different from the first reporter protein. Alternatively, a tau reporter cell or tau reporter non-human animal can comprise a first nucleic acid encoding a 4R tau isoform linked to a first reporter protein and a second nucleic acid encoding a 3R tau isoform linked to a second reporter protein. By “linked” it is meant that the tau isoform and the linked reporter protein are part of the same protein (i.e., a fusion protein) or are expressed from the same messenger RNA. In a fusion protein, the tau isoform and reporter protein can be directly fused to each other or can fused to each other via a linker. In a specific example, the tau isoform and the linked reporter protein are fused to each other via a linker. In a first example, the tau reporter cell or tau reporter non-human animal can comprise a first fusion protein comprising a 4R tau isoform fused to a first reporter protein and a second fusion protein comprising a 3R tau isoform fused to a second reporter protein that is different from the first reporter protein. For example, the cell or non-human animal can comprise a first nucleic acid (e.g., expression cassette) encoding the first fusion protein and a second nucleic acid (e.g., expression cassette) encoding the second fusion protein, wherein the cell or non-human animal expresses the first fusion protein and the second fusion protein. In a second example, the tau reporter cell or tau reporter non-human animal can comprise a first nucleic acid (e.g., expression cassette) comprising a coding sequence for a 4R tau isoform and a coding sequence for a first reporter protein and a second nucleic acid (e.g., expression cassette) comprising a coding sequence for a 3R tau isoform and a coding sequence for a second reporter protein that is different from the first reporter protein, wherein the coding sequence for the 4R tau isoform and the coding sequence for the first reporter protein are separated by the coding sequence for a first 2A peptide, and the coding sequence for the 3R tau isoform and the coding sequence for the second reporter protein are separated by the coding sequence for a second 2A peptide, and wherein the cell or non-human animal expresses the 4R tau isoform, the 3R tau isoform, the first reporter protein, and the second reporter protein.

As described, above, examples of 2A peptides that can be used include Thoseaasigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 9); P2A (ATNFSLLKQAGDVEENPGP; SEQ ID NO: 10); E2A (QCTNYALLKLAGDVESNPGP; SEQ ID NO: 11); and F2A (VKQTLNFDLLKLAGDVESNPGP; SEQ ID NO: 12). GSG residues can be added to the 5′ end of any of these peptides. In a specific example, P2A is used.

In a tau reporter cell or tau reporter non-human animal comprising the first nucleic acid and the second nucleic acid, such nucleic acids can be RNA (e.g., messenger RNA (mRNA)) or DNA, can be single-stranded or double-stranded, and can be linear or circular. DNA can be part of a vector, such as an expression vector or a targeting vector. The vector can also be a viral vector such as adenoviral, adeno-associated viral, lentiviral, and retroviral vectors.

Optionally, the nucleic acids can be codon-optimized for efficient translation into protein in a particular cell or organism. For example, the nucleic acid can be modified to substitute codons having a higher frequency of usage in a human cell, a non-human cell, a mammalian cell, a rodent cell, a mouse cell, a rat cell, a bacterial cell, a yeast cell, or any other host cell of interest, as compared to the naturally occurring polynucleotide sequence.

The nucleic acids or expression cassettes can be stably integrated into the genome (i.e., into a chromosome) of the cell or non-human animal or they can be located outside of a chromosome (e.g., extrachromosomally replicating DNA). The stably integrated expression cassettes or nucleic acids can be randomly integrated into the genome of the cell or non-human animal (i.e., transgenic), or they can be integrated into a predetermined region of the genome of the cell or non-human animal (i.e., knock-in). In one example, a nucleic acid or expression cassette is stably integrated into a safe harbor locus as described elsewhere herein. The target genomic locus at which a nucleic acid or expression cassette is stably integrated can be heterozygous for the nucleic acid or expression cassette or homozygous for the nucleic acid or expression cassette.

A nucleic acid or expression cassette described herein can be operably linked to any suitable promoter for expression in vivo within a non-human animal or in vitro or ex vivo within a cell. The non-human animal can be any suitable non-human animal as described elsewhere herein. As one example, a nucleic acid or expression cassette can be operably linked to an endogenous promoter at a target genomic locus, such as a Rosa26 promoter. Alternatively, a nucleic acid or expression cassette can be operably linked to an exogenous promoter, such as a constitutively active promoter (e.g., a CAG promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, or a zygote. Such promoters can be, for example, conditional promoters, inducible promoters, constitutive promoters, or tissue-specific promoters.

The expression cassettes described herein can be in any form. For example, an expression cassette can be in a vector or plasmid, such as a viral vector. The expression cassette can be operably linked to a promoter in an expression construct capable of directing expression of a protein or RNA. Alternatively, an expression cassette can be in a targeting vector. For example, the targeting vector can comprise homology arms flanking the expression cassette, wherein the homology arms are suitable for directing recombination with a desired target genomic locus to facilitate genomic integration and/or replacement of endogenous sequence.

The expression cassettes described herein can be in vitro, they can be within a cell (e.g., an embryonic stem cell) ex vivo (e.g., genomically integrated or extrachromosomal), or they can be in an organism (e.g., a non-human animal) in vivo (e.g., genomically integrated or extrachromosomal). If ex vivo, the expression cassette(s) can be in any type of cell from any organism, such as a totipotent cell such as an embryonic stem cell (e.g., a mouse or a rat embryonic stem cell) or an induced pluripotent stem cell (e.g., a human induced pluripotent stem cell). If in vivo, the expression cassette(s) can be in any type of organism (e.g., a non-human animal as described further elsewhere herein).

In a tau reporter cell or tau reporter non-human animal comprising a first nucleic acid (e.g., expression cassette) encoding a first fusion protein and a second nucleic acid (e.g., expression cassette) encoding a second fusion protein, the first fusion protein and the second fusion protein can be stably expressed in the cell or non-human animal. For example, the first nucleic acid (e.g., expression cassette) and the second nucleic acid (e.g., expression cassette) can be genomically integrated in the cell or non-human animal and operably linked to promoters active in the cell or non-human animal. For example, the first nucleic acid and the second nucleic acid can be genomically integrated in the germline of the non-human animal. Likewise, in a tau reporter cell or non-human animal comprising a first nucleic acid (e.g., expression cassette) comprising a coding sequence for a 4R tau isoform and a coding sequence for a first reporter protein (e.g., separated by a first 2A coding sequence) and a second nucleic acid (e.g., expression cassette) comprising a coding sequence for a 3R tau isoform and a coding sequence for a second reporter protein (e.g., separated by a second 2A coding sequence), the 4R tau isoform, the first reporter protein, the 3R tau isoform, and the second reporter protein can be stably expressed in the cell or non-human animal. For example, the first nucleic acid (e.g., expression cassette) and the second nucleic acid (e.g., expression cassette) can be genomically integrated in the cell or non-human animal and operably linked to promoters active in the cell or non-human animal. For example, the first nucleic acid and the second nucleic acid can be genomically integrated in the germline of the non-human animal. The nucleic acids (e.g., expression cassettes) can be randomly integrated in the genome of the cell or non-human animal, or they can be integrated into a target genomic locus, such as a safe harbor locus. Any target genomic locus capable of expressing a gene can be used. The promoter can be any suitable promoter. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter).

An example of a target genomic locus into which the nucleic acids or expression cassettes described herein can be stably integrated is a safe harbor locus in the genome of the cell or non-human animal. Safe harbor loci are described above. For example, the Rosa26 locus and its equivalent in humans offer an open chromatin configuration in all tissues and is ubiquitously expressed during embryonic development and in adults. In addition, the Rosa26 locus can be targeted with high efficiency, and disruption of the Rosa26 gene produces no overt phenotype. Other examples of safe harbor loci include CCR5, HPRT, AAVS1, and albumin. Biallelic targeting of safe harbor loci such as the Rosa26 locus has no negative consequences, so different genes or reporters can be targeted to the two Rosa26 alleles.

Nucleic acids (e.g., expression cassettes) integrated into a target genomic locus can be operably linked to an endogenous promoter at the target genomic locus or can be operably linked to an exogenous promoter that is heterologous to the target genomic locus.

Alternatively, the tau reporter cell or non-human animal can comprise a vector (e.g., a viral vector) comprising the first nucleic acid (e.g., expression cassette) and the second nucleic acid (e.g., expression cassette), or a first vector (e.g., a viral vector) comprising the first nucleic acid (e.g., expression cassette) and a second vector (e.g., a viral vector) comprising the second nucleic acid (e.g., expression cassette).

Vectors are described in more detail above. Some vectors may be circular. Alternatively, the vector may be linear. Non-limiting exemplary vectors include plasmids, phagemids, cosmids, artificial chromosomes, minichromosomes, transposons, viral vectors, and expression vectors. Viral vectors are described in more detail above. Numerous forms of viral vectors are known. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression.

In one example, the nucleic acid or expression construct is in an AAV vector. The AAV may be any suitable serotype and may be a single-stranded AAV (ssAAV) or a self-complementary AAV (scAAV). Such AAV vectors are described in more detail above.

The 4R and 3R tau isoforms in the tau reporter cell or non-human animal can be expressed at similar levels. For example, the 4R and 3R tau isoform mRNAs can have similar relative expression, and/or the 4R and 3R tau isoform proteins can have similar relative expression. In one example, mRNA levels of the 4R and 3R tau isoforms are within one cycle threshold (Ct) value of each other when measured by quantitative polymerase chain reaction (qPCR).

The 4R and 3R tau isoforms in the tau reporter cell or non-human animal can be from any animal or mammal, such as human, mouse, or rat. In a specific example, the 4R and 3R tau isoforms are human. The 4R and 3R tau isoforms can also be any combination of 4R and 3R tau isoforms, respectively. In one example, the 4R tau isoform is a 2N4R isoform. For example, the 4R tau isoform can comprise the sequence set forth in SEQ ID NO: 13, optionally wherein the 4R tau isoform is encoded by the sequence set forth in SEQ ID NO: 17. For example, in compositions in which the 4R tau isoform is fused to the first reporter protein, the fusion protein can comprise the sequence set forth in SEQ ID NO: 15, optionally wherein the fusion protein is encoded by the sequence set forth in SEQ ID NO: 19. In another example, the 4R tau isoform is a 1N4R isoform. For example, the 4R tau isoform can comprise the sequence set forth in SEQ ID NO: 23, 27, 31, or 47, optionally wherein the 4R tau isoform is encoded by the sequence set forth in SEQ ID NO: 25, 29, 33, or 48, respectively. For example, in compositions in which the 4R tau isoform is fused to the first reporter protein, the fusion protein can comprise the sequence set forth in SEQ ID NO: 35, optionally wherein the fusion protein is encoded by the sequence set forth in SEQ ID NO: 36. For example, in compositions in which the 4R tau isoform is fused to the first reporter protein, the fusion protein can comprise the sequence set forth in SEQ ID NO: 37, optionally wherein the fusion protein is encoded by the sequence set forth in SEQ ID NO: 38. For example, in compositions in which the 4R tau isoform is fused to the first reporter protein, the fusion protein can comprise the sequence set forth in SEQ ID NO: 39, optionally wherein the fusion protein is encoded by the sequence set forth in SEQ ID NO: 40.

In one example, the 3R tau isoform is a 2N3R isoform. For example, the 3R tau isoform can comprise the sequence set forth in SEQ ID NO: 14, optionally wherein the 3R tau isoform is encoded by the sequence set forth in SEQ ID NO: 18. For example, in compositions in which the 3R tau isoform is fused to the second reporter protein, the fusion protein can comprise the sequence set forth in SEQ ID NO: 16, optionally wherein the fusion protein is encoded by the sequence set forth in SEQ ID NO: 20. In another example, the 4R tau isoform is a 1N3R isoform. For example, the 3R tau isoform can comprise the sequence set forth in SEQ ID NO: 24, 28, 32, or 49, optionally wherein the 3R tau isoform is encoded by the sequence set forth in SEQ ID NO: 26, 30, 34, or 50, respectively. For example, in compositions in which the 3R tau isoform is fused to the second reporter protein, the fusion protein can comprise the sequence set forth in SEQ ID NO: 41, optionally wherein the fusion protein is encoded by the sequence set forth in SEQ ID NO: 42. For example, in compositions in which the 3R tau isoform is fused to the second reporter protein, the fusion protein can comprise the sequence set forth in SEQ ID NO: 43, optionally wherein the fusion protein is encoded by the sequence set forth in SEQ ID NO: 44. For example, in compositions in which the 3R tau isoform is fused to the second reporter protein, the fusion protein can comprise the sequence set forth in SEQ ID NO: 45, optionally wherein the fusion protein is encoded by the sequence set forth in SEQ ID NO: 46.

In one specific example, the 4R tau isoform is a 2N4R isoform, and the 3R tau isoform is a 2N3R isoform. For example, the 4R tau isoform can comprise the sequence set forth in SEQ ID NO: 13, and the 3R tau isoform can comprise the sequence set forth in SEQ ID NO: 14 (optionally encoded by SEQ ID NOS: 17 and 18, respectively). For example, the 4R tau isoform can be fused to the first reporter protein, and the fusion protein can comprise the sequence set forth in SEQ ID NO: 15, and the 3R tau isoform can be fused to the second reporter protein, and the fusion protein can comprise the sequence set forth in SEQ ID NO: 16 (optionally encoded by SEQ ID NOS: 19 and 20, respectively). In another specific example, the 4R tau isoform is a 1N4R isoform, and the 3R tau isoform is a 1N3R isoform. For example, the 4R tau isoform can comprise the sequence set forth in SEQ ID NO: 23, and the 3R tau isoform can comprise the sequence set forth in SEQ ID NO: 24 (optionally encoded by SEQ ID NOS: 25 and 26, respectively). For example, the 4R tau isoform can comprise the sequence set forth in SEQ ID NO: 27, and the 3R tau isoform can comprise the sequence set forth in SEQ ID NO: 28 (optionally encoded by SEQ ID NOS: 29 and 30, respectively). For example, the 4R tau isoform can comprise the sequence set forth in SEQ ID NO: 31, and the 3R tau isoform can comprise the sequence set forth in SEQ ID NO: 32 (optionally encoded by SEQ ID NOS: 33 and 34, respectively). For example, the 4R tau isoform can comprise the sequence set forth in SEQ ID NO: 47, and the 3R tau isoform can comprise the sequence set forth in SEQ ID NO: 49 (optionally encoded by SEQ ID NOS: 48 and 50, respectively). For example, the 4R tau isoform can be fused to the first reporter protein, and the fusion protein can comprise the sequence set forth in SEQ ID NO: 35, and the 3R tau isoform can be fused to the second reporter protein, and the fusion protein can comprise the sequence set forth in SEQ ID NO: 41 (optionally encoded by SEQ ID NOS: 36 and 42, respectively). For example, the 4R tau isoform can be fused to the first reporter protein, and the fusion protein can comprise the sequence set forth in SEQ ID NO: 37, and the 3R tau isoform can be fused to the second reporter protein, and the fusion protein can comprise the sequence set forth in SEQ ID NO: 43 (optionally encoded by SEQ ID NOS: 38 and 44, respectively). For example, the 4R tau isoform can be fused to the first reporter protein, and the fusion protein can comprise the sequence set forth in SEQ ID NO: 39, and the 3R tau isoform can be fused to the second reporter protein, and the fusion protein can comprise the sequence set forth in SEQ ID NO: 45 (optionally encoded by SEQ ID NOS: 40 and 46, respectively). In addition to the 2N4R/2N3R and 1N4R/1N3R combinations disclosed above, any other combination can be used (e.g., 0N4R/0N3R, 0N4R/1N3R, 0N4R/2N3R, 1N4R/0N3R, 1N4R/1N3R, 1N4R/2N3R, 2N4R/0N3R, 2N4R/1N3R, or 2N4R/2N3R).

The 4R and/or 3R tau isoforms can be wild type. Alternatively, the 4R and/or 3R tau isoforms can comprise a tau pathogenic mutation, such as a pro-aggregation mutation. Such a mutation can be, for example, a mutation that is associated with (e.g., segregates with) or causes a tauopathy. As one example, the mutation can be an aggregation-sensitizing mutation that sensitizes tau to seeding but does not result in tau readily aggregating on its own, or it can be a mutation that is known to cause aggregation of both 3R and 4R tau. For example, the tau mutation can be RSL, L237V, or G243V. By R5L mutation is meant the human tau R5L mutation or a corresponding mutation in another tau protein when optimally aligned with the human tau protein. By L237V mutation is meant the human tau L237V mutation or a corresponding mutation in another tau protein when optimally aligned with the human tau protein. By G243V mutation is meant the human tau G243V mutation or a corresponding mutation in another tau protein when optimally aligned with the human tau protein. In one example, both the 4R and 3R tau isoforms comprise a R5L mutation. In one example, both the 4R and 3R tau isoforms comprise a L237V mutation. In one example, both the 4R and 3R tau isoforms comprise a G243V mutation.

The first and second reporter proteins can be any suitable reporter proteins encoded by any suitable reporter gene. Reporter genes and reporter proteins are described in more detail above. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. In a specific example, the first and second reporter proteins are fluorescent reporter proteins. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods. In a specific example, the first and second reporter proteins are a yellow fluorescent protein (e.g., eYFP) and a red fluorescent protein (e.g., mCherry), respectively, or vice versa.

The tau reporter cells disclosed herein can be any type of cell and can be in vitro, ex vivo, or in vivo. A tau reporter cell line or population of cells can be a monoclonal cell line or population of cells. The cell can be from any source. For example, the cell can be a eukaryotic cell, an animal cell, a plant cell, or a fungal (e.g., yeast) cell. Such cells can be fish cells or bird cells, or such cells can be mammalian cells, such as human cells, non-human mammalian cells, rodent cells, mouse cells, or rat cells. Mammals include, for example, humans, non-human primates, monkeys, apes, cats dogs, horses, bulls, deer, bison, sheep, rodents (e.g., mice, rats, hamsters, guinea pigs), livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. In a specific example, the tau reporter cells are human cells (e.g., HEK293 cells).

The cell can be, for example, a totipotent cell or a pluripotent cell (e.g., an embryonic stem (ES) cell such as a rodent ES cell, a mouse ES cell, or a rat ES cell). Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cell can also be a primary somatic cell, or a cell that is not a primary somatic cell. Somatic cells can include any cell that is not a gamete, germ cell, gametocyte, or undifferentiated stem cell. The cell can also be a primary cell. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, somatic cells, hematopoietic cells, endothelial cells, epithelial cells, fibroblasts, mesenchymal cells, keratinocytes, melanocytes, monocytes, mononuclear cells, adipocytes, preadipocytes, neurons, glial cells, hepatocytes, skeletal myoblasts, and smooth muscle cells. For example, primary cells can be derived from connective tissues, muscle tissues, nervous system tissues, or epithelial tissues.

Such cells also include would normally not proliferate indefinitely but, due to mutation or alteration, have evaded normal cellular senescence and instead can keep undergoing division. Such mutations or alterations can occur naturally or be intentionally induced. Examples of immortalized cells include Chinese hamster ovary (CHO) cells, human embryonic kidney cells (e.g., HEK293 cells), and mouse embryonic fibroblast cells (e.g., 3T3 cells). Numerous types of immortalized cells are well known. Immortalized or primary cells include cells that are typically used for culturing or for expressing recombinant genes or proteins.

The cell can also be a differentiated cell, such as a neuronal cell (e.g., a human neuronal cell). The cell can be an isolated cell.

Non-human animals described herein can be any suitable non-human animal. The term “animal” includes any member of the animal kingdom, including, for example, mammals, fishes, reptiles, amphibians, birds, and worms. In a specific example, the non-human animal is a non-human mammal. Non-human mammals include, for example, non-human primates and rodents (e.g., mice and rats). The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Sv1m), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal wN, C57BL/6, C57BL/6J, C57BL/6ByJ, C57BL/6NJ, C57BL/10, C57BL/10ScSn, C57BL/10Cr, and C57BL/01a. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

### IV. Methods of Using Tau Reporter Cells and Tau Reporter Non-Human Animals

Various methods are provided for using the tau reporter cells and tau reporter non-human animals as described elsewhere herein. Such methods can be, for example, for assessing the activity of a tau-targeting reagent (e.g., a 4R-tau-targeting reagent). Such tau reporter cells and tau reporter non-human animals are particularly useful for distinguishing tau-targeting reagent targeting 4R tau specifically or selectively versus reagents targeting both 4R tau and 3R tau.

A. Methods of Assessing Activity of Tau-Targeting Reagents in Tau Reporter Cells

Various methods are provided for assessing the activity of tau-targeting reagent in the tau reporter cells described herein. Such methods can comprise: (a) administering the tau-targeting reagent to the cell; and (b) assessing the activity of the tau-targeting reagent in the cell. The assessing can be, for example, compared to a control tau reporter cell that was not administered the tau-targeting reagent or compared to the tau reporter cell prior to administration of the tau-targeting reagent.

Methods for assessing activity of the tau-targeting reagent are well-known and are provided elsewhere herein. In some methods, the assessing can comprise comprises measuring one or more of 4R tau messenger RNA expression, first reporter protein messenger RNA expression, and third reporter protein messenger RNA expression. Methods for assessing messenger RNA (mRNA) expression are provided elsewhere herein and are well-known.

In one example, the assessing comprises measuring 4R tau isoform mRNA expression and second reporter protein mRNA expression, wherein a larger relative decrease in 4R tau isoform mRNA expression compared to second reporter protein mRNA expression after administration of the tau-targeting reagent indicates that the tau-targeting reagent is a 4R-preferential tau targeting reagent. For example, the decrease in 4R tau isoform mRNA expression after administration of the tau-targeting reagent can be at least 50%, at least 55%, at least 60%, at least 65%, at least 70%, or at least 75%, and the decrease in second reporter protein mRNA expression after administration of the tau-targeting reagent can be no more than 40%, no more than 35%, no more than 30%, no more than 25%, no more than 20%, no more than 15%, no more than 10%, or no more than 5%. In one example, the decrease in 4R tau isoform mRNA expression after administration of the tau-targeting reagent can be at least 60%, and the decrease in second reporter protein mRNA expression after administration of the tau-targeting reagent can be no more than 30%. In another example, the decrease in 4R tau isoform mRNA expression after administration of the tau-targeting reagent can be at least 70%, and the decrease in second reporter protein mRNA expression after administration of the tau-targeting reagent can be no more than 30%. In one example, the decrease in 4R tau isoform mRNA expression after administration of the tau-targeting reagent can be at least 75%, and the decrease in second reporter protein mRNA expression after administration of the tau-targeting reagent can be no more than 10%.

In another example, the assessing comprises measuring first reporter protein mRNA expression and second reporter protein mRNA expression, wherein a larger relative decrease in first reporter protein mRNA expression compared to second reporter protein mRNA expression after administration of the tau-targeting reagent indicates that the tau-targeting reagent is a 4R-preferential tau targeting reagent. For example, the decrease in first reporter protein mRNA expression after administration of the tau-targeting reagent can be at least 50%, at least 55%, at least 60%, at least 65%, at least 70%, or at least 75%, and the decrease in second reporter protein mRNA expression after administration of the tau-targeting reagent can be no more than 40%, no more than 35%, no more than 30%, no more than 25%, no more than 20%, no more than 15%, no more than 10%, or no more than 5%. In one example, the decrease in first reporter protein mRNA expression after administration of the tau-targeting reagent can be at least 60%, and the decrease in second reporter protein mRNA expression after administration of the tau-targeting reagent can be no more than 30%. In another example, the decrease in first reporter protein mRNA expression after administration of the tau-targeting reagent can be at least 70%, and the decrease in second reporter protein mRNA expression after administration of the tau-targeting reagent can be no more than 30%. In one example, the decrease in first reporter protein mRNA expression after administration of the tau-targeting reagent can be at least 75%, and the decrease in second reporter protein mRNA expression after administration of the tau-targeting reagent can be no more than 10%.

In some methods, the assessing comprises measuring one or more of first reporter protein expression and second reporter protein expression. Methods for assessing protein expression are provided elsewhere herein and are well-known. For example, the assessing can comprise measuring first reporter protein expression and second reporter protein expression, wherein a larger relative decrease in first reporter protein expression compared to second reporter protein expression after administration of the tau-targeting reagent indicates that the tau-targeting reagent is a 4R-preferential tau targeting reagent. For example, the decrease in first reporter protein expression after administration of the tau-targeting reagent can be at least 50%, at least 55%, at least 60%, at least 65%, at least 70%, or at least 75%, and the decrease in second reporter protein expression after administration of the tau-targeting reagent can be no more than 40%, no more than 35%, no more than 30%, no more than 25%, no more than 20%, no more than 15%, no more than 10%, or no more than 5%. In one example, the decrease in first reporter protein expression after administration of the tau-targeting reagent can be at least 60%, and the decrease in second reporter protein expression after administration of the tau-targeting reagent can be no more than 30%. In another example, the decrease in first reporter protein expression after administration of the tau-targeting reagent can be at least 70%, and the decrease in second reporter protein expression after administration of the tau-targeting reagent can be no more than 30%. In one example, the decrease in first reporter protein expression after administration of the tau-targeting reagent can be at least 75%, and the decrease in second reporter protein expression after administration of the tau-targeting reagent can be no more than 10%.

In a specific example, the first reporter protein is a first fluorescent reporter protein, and the second reporter protein is a second fluorescent reporter protein, and the assessing in step (b) comprises immunofluorescence staining or flow cytometry. In some methods, the assessing comprises assessing tau hyperphosphorylation or tau aggregation.

If the tau-targeting reagent is a genome editing reagent (e.g., a nuclease agent), such methods can comprise assessing modification of the nucleic acid encoding the tau 4R or tau 3R isoform. As one example, the assessing can comprise measuring non-homologous end joining (NHEJ) activity. This can comprise, for example, measuring the frequency of insertions or deletions within the target region. For example, the assessing can comprise sequencing the nucleic acid encoding the tau 4R or tau 3R isoform (e.g., next-generation sequencing).

As one specific example, if the tau-targeting reagent is a genome editing reagent (e.g., a nuclease agent), percent editing (e.g., total number of insertions or deletions observed over the total number of sequences read in the PCR reaction from a pool of lysed cells) of the target nucleic acid can be assessed.

The tau-targeting reagent can be a tau-targeting antibody or antigen-binding protein (e.g., an intrabody) or any other large molecule or small molecule that targets tau protein. Alternatively, the tau-targeting reagent can be any biological or chemical agent that targets the MAPT locus (the MAPT gene), the MAPT mRNA, or the tau protein. Examples of tau-targeting reagents are disclosed elsewhere herein.

Such tau-targeting reagents can be administered by any delivery method/vehicle (e.g., adenovirus, lentivirus, AAV, or LNP). Means of delivering complexes and molecules are disclosed in more detail elsewhere herein. In particular methods, the reagents are delivered via AAV-mediated delivery or lentiviral-mediated delivery. In other particular methods, the reagents are delivered by LNP-mediated delivery. The dose can be any suitable dose.

B. Methods of Assessing Activity of Tau-Targeting Reagents in Tau Reporter Non-Human Animals

Various methods are provided for assessing the activity of tau-targeting reagent in the tau reporter non-human animals described herein. Such methods can comprise: (a) administering the tau-targeting reagent to the non-human animal; and (b) assessing the activity of the tau-targeting reagent in the non-human animal. The assessing can be, for example, compared to a control tau reporter non-human animal that was not administered the tau-targeting reagent or compared to the tau reporter non-human animal prior to administration of the tau-targeting reagent.

Methods for assessing activity of the tau-targeting reagent are well-known and are provided elsewhere herein. Assessment of activity can be in any cell type, any tissue type, or any organ type as disclosed elsewhere herein. In some methods, assessment of activity is in neurons. In some methods, the assessing can comprise comprises measuring one or more of 4R tau mRNA expression, first reporter protein mRNA expression, and third reporter protein mRNA expression. For example, mRNA levels can be measured in a particular cell, tissue, or organ type (e.g., neurons). Methods for assessing mRNA expression are provided elsewhere herein and are well-known.

In one example, the assessing comprises measuring 4R tau isoform mRNA expression and second reporter protein mRNA expression, wherein a larger relative decrease in 4R tau isoform mRNA expression compared to second reporter protein mRNA expression after administration of the tau-targeting reagent indicates that the tau-targeting reagent is a 4R-preferential tau targeting reagent. For example, the decrease in 4R tau isoform mRNA expression after administration of the tau-targeting reagent can be at least 50%, at least 55%, at least 60%, at least 65%, at least 70%, or at least 75%, and the decrease in second reporter protein mRNA expression after administration of the tau-targeting reagent can be no more than 40%, no more than 35%, no more than 30%, no more than 25%, no more than 20%, no more than 15%, no more than 10%, or no more than 5%. In one example, the decrease in 4R tau isoform mRNA expression after administration of the tau-targeting reagent can be at least 60%, and the decrease in second reporter protein mRNA expression after administration of the tau-targeting reagent can be no more than 30%. In another example, the decrease in 4R tau isoform mRNA expression after administration of the tau-targeting reagent can be at least 70%, and the decrease in second reporter protein mRNA expression after administration of the tau-targeting reagent can be no more than 30%. In one example, the decrease in 4R tau isoform mRNA expression after administration of the tau-targeting reagent can be at least 75%, and the decrease in second reporter protein mRNA expression after administration of the tau-targeting reagent can be no more than 10%.

In another example, the assessing comprises measuring first reporter protein mRNA expression and second reporter protein mRNA expression, wherein a larger relative decrease in first reporter protein mRNA expression compared to second reporter protein mRNA expression after administration of the tau-targeting reagent indicates that the tau-targeting reagent is a 4R-preferential tau targeting reagent. For example, the decrease in first reporter protein mRNA expression after administration of the tau-targeting reagent can be at least 50%, at least 55%, at least 60%, at least 65%, at least 70%, or at least 75%, and the decrease in second reporter protein mRNA expression after administration of the tau-targeting reagent can be no more than 40%, no more than 35%, no more than 30%, no more than 25%, no more than 20%, no more than 15%, no more than 10%, or no more than 5%. In one example, the decrease in first reporter protein mRNA expression after administration of the tau-targeting reagent can be at least 60%, and the decrease in second reporter protein mRNA expression after administration of the tau-targeting reagent can be no more than 30%. In another example, the decrease in first reporter protein mRNA expression after administration of the tau-targeting reagent can be at least 70%, and the decrease in second reporter protein mRNA expression after administration of the tau-targeting reagent can be no more than 30%. In one example, the decrease in first reporter protein mRNA expression after administration of the tau-targeting reagent can be at least 75%, and the decrease in second reporter protein mRNA expression after administration of the tau-targeting reagent can be no more than 10%.

In some methods, the assessing comprises measuring one or more of first reporter protein expression and second reporter protein expression. Methods for assessing protein expression are provided elsewhere herein and are well-known. For example, the assessing can comprise measuring first reporter protein expression and second reporter protein expression, wherein a larger relative decrease in first reporter protein expression compared to second reporter protein expression after administration of the tau-targeting reagent indicates that the tau-targeting reagent is a 4R-preferential tau targeting reagent. For example, the decrease in first reporter protein expression after administration of the tau-targeting reagent can be at least 50%, at least 55%, at least 60%, at least 65%, at least 70%, or at least 75%, and the decrease in second reporter protein expression after administration of the tau-targeting reagent can be no more than 40%, no more than 35%, no more than 30%, no more than 25%, no more than 20%, no more than 15%, no more than 10%, or no more than 5%. In one example, the decrease in first reporter protein expression after administration of the tau-targeting reagent can be at least 60%, and the decrease in second reporter protein expression after administration of the tau-targeting reagent can be no more than 30%. In another example, the decrease in first reporter protein expression after administration of the tau-targeting reagent can be at least 70%, and the decrease in second reporter protein expression after administration of the tau-targeting reagent can be no more than 30%. In one example, the decrease in first reporter protein expression after administration of the tau-targeting reagent can be at least 75%, and the decrease in second reporter protein expression after administration of the tau-targeting reagent can be no more than 10%.

In a specific example, the first reporter protein is a first fluorescent reporter protein, and the second reporter protein is a second fluorescent reporter protein, and the assessing in step (b) comprises immunofluorescence staining or flow cytometry. In some methods, the assessing comprises assessing tau hyperphosphorylation or tau aggregation.

If the tau-targeting reagent is a genome editing reagent (e.g., a nuclease agent), such methods can comprise assessing modification of the nucleic acid encoding the tau 4R or tau 3R isoform. As one example, the assessing can comprise measuring non-homologous end joining (NHEJ) activity. This can comprise, for example, measuring the frequency of insertions or deletions within the target region. For example, the assessing can comprise sequencing the nucleic acid encoding the tau 4R or tau 3R isoform (e.g., next-generation sequencing). Assessment can comprise isolating a target organ or tissue (e.g., neurons) from the non-human animal and assessing modification of nucleic acid encoding the tau 3R or tau 4R isoform in the target organ or tissue. Similarly, assessment can comprise isolating a non-target organ or tissue (e.g., two or more non-target organs or tissues) from the non-human animal and assessing modification of nucleic acid encoding the tau 3R or tau 4R isoform in the non-target organ or tissue.

As one specific example, if the tau-targeting reagent is a genome editing reagent (e.g., a nuclease agent), percent editing (e.g., total number of insertions or deletions observed over the total number of sequences read in the PCR reaction from a pool of lysed cells) of the target nucleic acid can be assessed.

The tau-targeting reagent can be a tau-targeting antibody or antigen-binding protein (e.g., an intrabody) or any other large molecule or small molecule that targets tau protein. Alternatively, the tau-targeting reagent can be any biological or chemical agent that targets the MAPT locus (the MAPT gene), the MAPT mRNA, or the tau protein. Examples of tau-targeting reagents are disclosed elsewhere herein.

Such tau-targeting reagents can be administered by any delivery method/vehicle (e.g., adenovirus, lentivirus, AAV, LNP, or injection) and by any route of administration (e.g., intravitreal injection or intracameral injection). Means of delivering complexes and molecules and routes of administration are disclosed in more detail elsewhere herein. In particular methods, the reagents delivered via AAV-mediated delivery or lentiviral-mediated delivery. In other particular methods, the reagents are delivered by LNP-mediated delivery. The dose can be any suitable dose.

C. Tau-Targeting Reagents

A tau-targeting reagent can be any reagent that targets a tau protein, a MAPT gene, or a MAPT mRNA (e.g., a human tau protein, a human MAPT gene, or a human MAPT mRNA). A tau-targeting reagent can be, for example, a known tau-targeting reagent, can be a putative tau-targeting reagent (e.g., candidate reagents designed to target a tau protein, a MAPT gene, or a MAPT mRNA), or can be a reagent being screened for tau-targeting activity.

For example, a tau-targeting reagent can be an antigen-binding protein targeting an epitope of a tau protein. The term “antigen-binding protein” includes any protein that binds to an antigen. Examples of antigen-binding proteins include an antibody, an antigen-binding fragment of an antibody, a multispecific antibody (e.g., a bi-specific antibody), an scFV, a bis-scFV, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes). In a specific example, the antigen-binding protein is an intrabody. An intrabody is an antibody that has been designed to be expressed intracellularly. Other tau-targeting reagents include small molecules targeting a tau protein, MAPT gene, or MAPT mRNA.

Other tau-targeting reagents can include genome editing reagents such as a nuclease agent (e.g., a Clustered Regularly Interspersed Short Palindromic Repeats (CRISPR)/CRISPR-associated (Cas) (CRISPR/Cas) nuclease, a zinc finger nuclease (ZFN), or a Transcription Activator-Like Effector Nuclease (TALEN)) that cleaves a recognition site within the MAPT gene. Likewise, a tau-targeting reagent can be an exogenous donor nucleic acid (e.g., a targeting vector or single-stranded oligodeoxynucleotide (ssODN)) designed to recombine with the MAPT gene.

Other tau-targeting reagents can include RNAi agents. An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to: short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Other tau-targeting reagents can include antisense oligonucleotides (ASOs). Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H.

Other tau-targeting reagents include small-molecule reagents.

D. Administering Tau-Targeting Reagents to Cells or Non-Human Animals

The methods disclosed herein can comprise introducing into a cell or non-human animal various molecules (e.g., tau-targeting reagents), including nucleic acids, proteins, nucleic-acid-protein complexes, protein complexes, or small molecules. “Introducing” includes presenting to the cell or non-human animal the molecule (e.g., nucleic acid or protein) in such a manner that it gains access to the interior of the cell or to the interior of cells within the non-human animal. The introducing can be accomplished by any means, and two or more of the components (e.g., two of the components, or all of the components) can be introduced into the cell or non-human animal simultaneously or sequentially in any combination. In addition, two or more of the components can be introduced into the cell or non-human animal by the same delivery method/vehicle or different delivery methods/vehicles. Similarly, two or more of the components can be introduced into a non-human animal by the same route of administration or different routes of administration.

Molecules (e.g., Cas proteins or guide RNAs or RNAi agents or ASOs) introduced into the cell or non-human animal can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or non-human animal. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4): 1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a protein or a polynucleotide encoding a protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359.

Other methods for introducing molecules (e.g., nucleic acid or proteins) into a cell or non-human animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., adeno-associated virus (AAV)-mediated delivery or lentivirus-mediated delivery), and lipid-nanoparticle-mediated delivery.

Introduction of nucleic acids can also be accomplished by virus-mediated delivery, such as adenovirus-mediated delivery, AAV-mediated delivery (e.g., AAV-PhPeB), or lentivirus-mediated delivery. Other exemplary viruses/viral vectors include retroviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Exemplary viral titers (e.g., AAV titers) include 10¹², 10¹³, 10¹⁴, 10¹⁵, and 10¹⁶ vector genomes/mL. Other exemplary viral titers (e.g., AAV titers) include about 10¹², about 10¹³, about 10¹⁴, about 10¹⁵, and about 10¹⁶ vector genomes(vg)/kg of body weight.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8. In a specific example, an AAV-PhPeB serotype is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

Introduction of nucleic acids and proteins can also be accomplished by lipid nanoparticle (LNP)-mediated delivery. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as 5010, 5024, 5027, 5031, or 5033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Reports 22:1-9 and WO 2017/173054 A1, each of which is herein incorporated by reference in its entirety for all purposes.

Exemplary dosing of LNPs includes, for example, about 0.1, about 0.25, about 0.3, about 0.5, about 1, about 2, about 3, about 4, about 5, about 6, about 8, or about 10 mg/kg (mpk) with respect to total RNA cargo content. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions being administered can be formulated using one or more physiologically and pharmaceutically acceptable carriers, diluents, excipients or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the administered molecules and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

E. Measuring Delivery, Activity, or Efficacy of Tau-Targeting Reagents Agents

The methods disclosed herein can further comprise detecting or measuring activity of tau-targeting reagents.

If the tau-targeting reagent is a genome editing reagent (e.g., CRISPR/Cas designed to target a nucleic acid encoding a tau 4R isoform), the measuring can comprise assessing the targeted nucleic acid for modifications. Various methods can be used to identify cells having a targeted genetic modification. The screening can comprise a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, herein incorporated by reference in its entirety for all purposes). Next-generation sequencing (NGS) can also be used for screening. Next-generation sequencing can also be referred to as “NGS” or “massively parallel sequencing” or “high throughput sequencing.” NGS can be used as a screening tool in addition to the MOA assays to define the exact nature of the targeted genetic modification and whether it is consistent across cell types or tissue types or organ types.

Assessing modification in a non-human animal can be in any cell type from any tissue or organ. For example, the assessment can be in multiple cell types from the same tissue or organ or in cells from multiple locations within the tissue or organ. This can provide information about which cell types within a target tissue or organ are being targeted or which sections of a tissue or organ are being reached by the tau-targeting reagent. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

If the reagent is designed to inactivate the nucleic acid encoding the tau 4R or tau 3R isoform, affect expression of the tau 4R or tau 3R isoform, prevent translation of the tau 4R or tau 3R isoform mRNA, or clear the tau 4R or tau 3R isoform protein, the measuring can comprise assessing tau 4R isoform, tau 3R isoform, first reporter protein, or second reporter protein mRNA or protein expression. This measuring can be, for example, within neurons.

Production of the tau 4R or tau 3R isoform protein can be assessed by any known means. For example, expression can be assessed by measuring levels of the encoded mRNA in the non-human animal or levels of the encoded protein in the non-human animal using known assays. For example, the measuring can be to determine if the tau-targeting reagent reduces tau 4R or tau 3R isoform levels in the non-human animal.

The assessing in a non-human animal can be in any cell type from any tissue or organ. For example, the assessment can be in multiple cell types from the same tissue or organ (e.g., central nervous system) or in cells from multiple locations within the tissue or organ. This can provide information about which cell types within a target tissue or organ are being targeted or which sections of a tissue or organ are being reached by the tau-targeting reagent. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

One example of an assay that can be used are the RNASCOPE™ and BASESCOPE™ RNA in situ hybridization (ISH) assays, which are methods that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a ZZ probe, and it can differentially detect single nucleotide edits and mutations in intact fixed tissue.

The assessment of any of these phenotypes can be done in comparison to a control cell or non-human animal. The control cells or non-human animals can be, for example, the same age as the test cell or non-human animal and/or the same sex as the test cell or non-human animal. The assessment of any of these phenotypes can also be done in comparison to a control cell or non-human animal that is identical to the test cell non-human animal except not treated with the tau-targeting reagent. The assessment of any of these phenotypes can also be done in comparison to the test cell or test non-human animal prior to administration of the tau-targeting reagent to the test cell non-human animal.

The assessment of any of these phenotypes can be in a single cell or non-human animal and assessing changes in that cell or non-human animal. Alternatively, the assessment can be in a population of cells or non-human animals and comparing, for example, the percentage of cells or non-human animals having a particular phenotype.

### V. Methods of Making Tau Reporter Cells and Tau Reporter Non-Human Animals

The tau reporter cells and non-human animals disclosed herein can be generated by any known means. For example, the tau reporter cells and non-human animals disclosed herein can be generated by introducing a 4R tau isoform linked to a first reporter protein and a 3R tau isoform linked to a second reporter protein, or a first nucleic acid encoding the 4R tau isoform linked to the first reporter protein and a second nucleic acid encoding the 3R tau isoform linked to the second reporter protein into a cell or non-human animal. A 4R tau isoform linked to a first reporter protein and a 3R tau isoform linked to a second reporter protein, or a first nucleic acid encoding the 4R tau isoform linked to the first reporter protein and a second nucleic acid encoding the 3R tau isoform linked to the second reporter protein can be introduced into the cell or non-human animal in any form (e.g., DNA, RNA, or protein) by any known means. “Introducing” includes presenting to the cell or non-human animal the nucleic acid or protein in such a manner that the sequence gains access to the interior of the cell or to the interior of cells within the non-human animal. The methods provided herein do not depend on a particular method for introducing a nucleic acid or protein into the cell or non-human animal, only that the nucleic acid or protein gains access to the interior of a least one cell. Methods for introducing nucleic acids and proteins into various cell types are known and include, for example, stable transfection methods, transient transfection methods, and virus-mediated methods. Optionally, targeting vectors can be used.

Transfection protocols as well as protocols for introducing nucleic acids or proteins into cells or non-human animals may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. USA 74 (4): 1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell or a non-human animal can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of nucleic acids or proteins into a cell can also be accomplished by microinjection. Microinjection of an mRNA is preferably into the cytoplasm (e.g., to deliver mRNA directly to the translation machinery), while microinjection of a protein or a DNA encoding a protein is preferably into the nucleus. Alternatively, microinjection can be carried out by injection into both the nucleus and the cytoplasm: a needle can first be introduced into the nucleus and a first amount can be injected, and while removing the needle from the cell a second amount can be injected into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); Meyer et al. (2010) Proc. Natl. Acad. Sci. USA 107:15022-15026 and Meyer et al. (2012) Proc. Natl. Acad. Sci. USA 109:9354-9359.

Other methods for introducing nucleic acid or proteins into a cell or a non-human animal can include, for example, vector delivery, particle-mediated delivery, exosome-mediated delivery, lipid-nanoparticle-mediated delivery, cell-penetrating-peptide-mediated delivery, or implantable-device-mediated delivery. Methods of administering nucleic acids or proteins to a subject to modify cells in vivo are disclosed elsewhere herein.

In one example, a first nucleic acid encoding the 4R tau isoform linked to the first reporter protein and a second nucleic acid encoding the 3R tau isoform linked to the second reporter protein can be introduced via viral transduction such as lentiviral transduction or AAV transduction.

Screening for cells or non-human animals comprising the 4R tau isoform linked to the first reporter protein and the 3R tau isoform linked to the second reporter can be performed by any known means.

As one example, reporter genes can be used to screen for cells or non-human animals that have the 4R tau isoform linked to the first reporter protein and the 3R tau isoform linked to the second reporter. Exemplary reporter genes include those encoding luciferase, 0-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. For example, if the first reporter and the second reporter are fluorescent proteins (e.g., mCherry and eYFP), cells comprising these reporters can be selected by flow cytometry to select for dual-positive cells. The dual-positive cells can then be combined to generate a polyclonal line, or monoclonal lines can be generated from single dual-positive cells.

As another example, selection markers can be used to screen for cells that have the 4R tau isoform linked to the first reporter protein and the 3R tau isoform linked to the second reporter. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k). Another exemplary selection marker is bleomycin resistance protein, encoded by the Sh ble gene (Streptoalloteichus hindustanus bleomycin gene), which confers zeocin (phleomycin D1) resistance.

Various other methods are provided for making a non-human animal comprising a 4R tau isoform linked to a first reporter protein and a 3R tau isoform linked to a second reporter protein, or a first nucleic acid encoding the 4R tau isoform linked to the first reporter protein and a second nucleic acid encoding the 3R tau isoform linked to the second reporter protein. Any convenient method or protocol for producing a genetically modified organism is suitable for producing such a genetically modified non-human animal. See, e.g., Poueymirou et al. (2007) Nat. Biotechnol. 25(1):91-99; U.S. Pat. Nos. 7,294,754; 7,576,259; 7,659,442; 8,816,150; 9,414,575; 9,730,434; and 10,039,269, each of which is herein incorporated by reference in its entirety for all purposes (describing mouse ES cells and the VELOCIMOUSE® method for making a genetically modified mouse). See also US 2014/0235933 A1, US 2014/0310828 A1, each of which is herein incorporated by reference in its entirety for all purposes (describing rat ES cells and methods for making a genetically modified rat). See also Cho et al. (2009) Curr. Protoc. Cell. Biol. 42:19.11.1-19.11.22 (doi: 10.1002/0471143030.cb1911s42) and Gama Sosa et al. (2010) Brain Struct. Funct. 214(2-3):91-109, each of which is herein incorporated by reference in its entirety for all purposes.

For example, a method of producing a tau reporter non-human animal as described herein can comprise: (1) providing a pluripotent cell (e.g., an embryonic stem (ES) cell such as a mouse ES cell or a rat ES cell) comprising the first nucleic acid encoding the 4R tau isoform linked to the first reporter protein and the second nucleic acid encoding the 3R tau isoform linked to the second reporter protein; (2) introducing the genetically modified pluripotent cell into a non-human animal host embryo; and (3) gestating the host embryo in a surrogate mother.

As another example, a method of producing a tau reporter non-human animal as described herein can comprise: (1) modifying the genome of a pluripotent cell (e.g., an embryonic stem (ES) cell such as a mouse ES cell or a rat ES cell) to comprise the first nucleic acid encoding the 4R tau isoform linked to the first reporter protein and the second nucleic acid encoding the 3R tau isoform linked to the second reporter protein; (2) identifying or selecting the genetically modified pluripotent cell comprising the first nucleic acid encoding the 4R tau isoform linked to the first reporter protein and the second nucleic acid encoding the 3R tau isoform linked to the second reporter protein; (3) introducing the genetically modified pluripotent cell into a non-human animal host embryo; and (4) gestating the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an FO non-human animal. The surrogate mother can then produce an FO generation non-human animal comprising the 4R tau isoform linked to a first reporter protein and the 3R tau isoform linked to a second reporter protein, or the first nucleic acid encoding the 4R tau isoform linked to the first reporter protein and the second nucleic acid encoding the 3R tau isoform linked to the second reporter protein. The non-human animal can be capable of transmitting the first nucleic acid encoding the 4R tau isoform linked to the first reporter protein and the second nucleic acid encoding the 3R tau isoform linked to the second reporter protein through the germline.

Alternatively, the method of producing the tau reporter non-human animals described elsewhere herein can comprise: (1) modifying the genome of a one-cell stage embryo to comprise the first nucleic acid encoding the 4R tau isoform linked to the first reporter protein and the second nucleic acid encoding the 3R tau isoform linked to the second reporter protein using the methods described above for modifying pluripotent cells; (2) selecting the genetically modified embryo; and (3) gestating the genetically modified embryo in a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The modified cell or one-cell stage embryo can be generated, for example, through recombination by (a) introducing into the cell one or more exogenous donor nucleic acids (e.g., targeting vectors) comprising an insert nucleic acid flanked, for example, by 5′ and 3′ homology arms corresponding to 5′ and 3′ target sites (e.g., target sites flanking the endogenous sequences intended for deletion and replacement with the insert nucleic acid), wherein the insert nucleic acid(s) comprises the first nucleic acid encoding the 4R tau isoform linked to the first reporter protein and the second nucleic acid encoding the 3R tau isoform linked to the second reporter protein; and (b) identifying at least one cell comprising in its genome the first nucleic acid encoding the 4R tau isoform linked to the first reporter protein and the second nucleic acid encoding the 3R tau isoform linked to the second reporter protein. Likewise, a modified non-human animal genome or humanized non-human animal target locus can be generated, for example, through recombination by (a) contacting the genome or gene with one or more exogenous donor nucleic acids (e.g., targeting vectors) comprising 5′ and 3′ homology arms corresponding to 5′ and 3′ target sites (e.g., target sites flanking the endogenous sequences intended for deletion and replacement with one or more insert nucleic acids (e.g., the first nucleic acid encoding the 4R tau isoform linked to the first reporter protein and the second nucleic acid encoding the 3R tau isoform linked to the second reporter protein). Optionally, a nuclease agent targeting the endogenous sequences intended for deletion can be introduced together with the exogenous donor nucleic acid. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA. In another example, the nuclease comprises a Cas9 protein and two or more, three or more, or four or more guide RNAs.

The exogenous donor nucleic acids can be for non-homologous-end-joining-mediated insertion or homologous recombination. Exogenous donor nucleic acids can comprise deoxyribonucleic acid (DNA) or ribonucleic acid (RNA), they can be single-stranded or double-stranded, and they can be in linear or circular form. For example, a donor nucleic acid can be a single-stranded oligodeoxynucleotide (ssODN). Exogenous donor nucleic acids can also comprise a heterologous sequence that is not present at an untargeted endogenous locus. For example, an exogenous donor nucleic acid can comprise a selection cassette, such as a selection cassette flanked by recombinase recognition sites.

In cells other than one-cell stage embryos, the exogenous donor nucleic acid can be a “large targeting vector” or “LTVEC,” which includes targeting vectors that comprise homology arms that correspond to and are derived from nucleic acid sequences larger than those typically used by other approaches intended to perform homologous recombination in cells. See, e.g., US 2004/0018626; WO 2013/163394; U.S. Pat. Nos. 9,834,786; 10,301,646; WO 2015/088643; U.S. Pat. Nos. 9,228,208; 9,546,384; 10,208,317; and US 2019-0112619, each of which is herein incorporated by reference in its entirety for all purposes. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

The methods can further comprise identifying a cell or animal having a modified target genomic locus. Various methods can be used to identify cells and animals having a targeted genetic modification. The screening step can comprise, for example, a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

The various methods provided herein allow for the generation of a genetically modified non-human FO animal wherein the cells of the genetically modified FO animal comprise the first nucleic acid encoding the 4R tau isoform linked to the first reporter protein and the second nucleic acid encoding the 3R tau isoform linked to the second reporter protein. It is recognized that depending on the method used to generate the FO animal, the number of cells within the FO animal that have the first nucleic acid encoding the 4R tau isoform linked to the first reporter protein and the second nucleic acid encoding the 3R tau isoform linked to the second reporter protein will vary. With mice, for example, the introduction of the donor ES cells into a pre-morula stage embryo from the mouse (e.g., an 8-cell stage mouse embryo) via, for example, the VELOCIMOUSE® method allows for a greater percentage of the cell population of the FO mouse to comprise cells having the targeted genetic modification. For example, at least 50%, at least 60%, at least 65%, at least 70%, at least 75%, at least 85%, at least 86%, at least 87%, at least 87%, at least 88%, at least 89%, at least 90%, at least 91%, at least 92%, at least 93%, at least 94%, at least 95%, at least 96%, at least 97%, at least 98%, at least 99% or 100% of the cellular contribution of the non-human FO animal can comprise a cell population having the targeted modification. The cells of the genetically modified FO animal can be heterozygous for the first nucleic acid encoding the 4R tau isoform linked to the first reporter protein and the second nucleic acid encoding the 3R tau isoform linked to the second reporter protein or can be homozygous for the first nucleic acid encoding the 4R tau isoform linked to the first reporter protein and the second nucleic acid encoding the 3R tau isoform linked to the second reporter protein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ ID NO | Type | Description |
|---|---|---|
| 1 | RNA | mCherry siRNA HAIJE-00013 - Sense Strand |
| 2 | RNA | mCherry siRNA HAIJE-00013 - Antisense Strand |
| 3 | RNA | mCherry siRNA HAIJE-00015 - Sense Strand |
| 4 | RNA | mCherry siRNA HAIJE-00015 - Antisense Strand |
| 5 | RNA | mCherry siRNA HAIJE-00017 - Sense Strand |
| 6 | RNA | mCherry siRNA HAIJE-00017 - Antisense Strand |
| 7 | RNA | YFP siRNA P-002048-01 - Sense Strand |
| 8 | RNA | YFP siRNA P-002048-01 - Antisense Strand |
| 9 | Protein | T2A |
| 10 | Protein | P2A |
| 11 | Protein | E2A |
| 12 | Protein | F2A |
| 13 | Protein | 2N4R Tau |
| 14 | Protein | 2N3R Tau |
| 15 | Protein | 2N4R-Tau-eYFP Fusion Protein |
| 16 | Protein | 2N3R-Tau-mCherry Fusion Protein |
| 17 | DNA | 2N4R Tau Coding Sequence |
| 18 | DNA | 2N3R Tau Coding Sequence |
| 19 | DNA | 2N4R-Tau-eYFP Fusion Protein |
| | | Coding Sequence |
| 20 | DNA | 2N3R-Tau-mCherry Fusion Protein |
| | | Coding Sequence |
| 21 | Protein | Tau R2 Domain |
| 22 | DNA | Tau R2 Domain Coding Sequence |
| 23 | Protein | 1N4R Tau R5L |
| 24 | Protein | 1N3R Tau R5L |
| 25 | DNA | 1N4R Tau R5L Coding Sequence |
| 26 | DNA | 1N3R Tau R5L Coding Sequence |
| 27 | Protein | 1N4R Tau L237V |
| 28 | Protein | 1N3R Tau L237V |
| 29 | DNA | 1N4R Tau L237V Coding Sequence |
| 30 | DNA | 1N3R Tau L237V Coding Sequence |
| 31 | Protein | 1N4R Tau G243V |
| 32 | Protein | 1N3R Tau G243V |
| 33 | DNA | 1N4R Tau G243V Coding Sequence |
| 34 | DNA | 1N3R Tau G243V Coding Sequence |
| 35 | Protein | 1N4R Tau R5L-P2A-eYFP |
| 36 | DNA | 1N4R Tau R5L-P2A-eYFP Coding Sequence |
| 37 | Protein | 1N4R Tau L237V-P2A-eYFP |
| 38 | DNA | 1N4R Tau L237V-P2A-eYFP |
| | | Coding Sequence |
| 39 | Protein | 1N4R Tau G243V-P2A-eYFP |
| 40 | DNA | 1N4R Tau G243V-P2A-eYFP |
| | | Coding Sequence |
| 41 | Protein | 1N3R Tau R5L-P2A-mCherry |
| 42 | DNA | 1N3R Tau R5L-P2A-mCherry |
| | | Coding Sequence |
| 43 | Protein | 1N3R Tau L237V-P2A-mCherry |
| 44 | DNA | 1N3R Tau L237V-P2A-mCherry |
| | | Coding Sequence |
| 45 | Protein | 1N3R Tau G243V-P2A-mCherry |
| 46 | DNA | 1N3R Tau G243V-P2A-mCherry |
| | | Coding Sequence |
| 47 | Protein | 1N4R Tau |
| 48 | DNA | 1N4R Tau Coding Sequence |
| 49 | Protein | 1N3R Tau |
| 50 | DNA | 1N3R Tau Coding Sequence |

### EXAMPLES

### Example 1. Development of Cellular Screening Assay to Distinguish Reagents Targeting 4R Versus 3R Tau

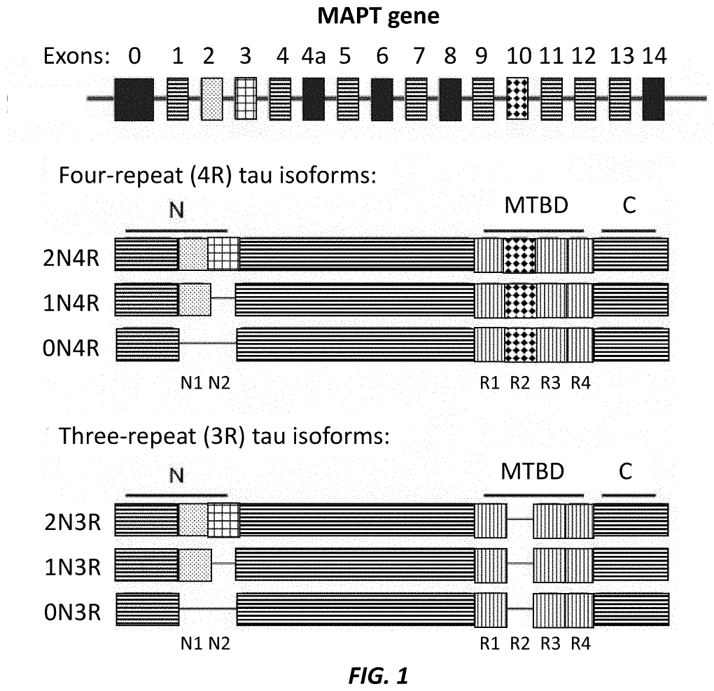

The human MAPT gene encodes 6 different isoforms of tau: 2N4R, 1N4R, 0N4R, 2N3R, 1N3R, 0N3R. See FIG. 1. The difference between transcripts containing the 4R (4 repeat) versus 3R (3 repeat) tau is based on the inclusion (4R) or exclusion (3R) of exon 10. Humans normally express equal ratios of 3R and 4R tau. In some tauopathies, such as Alzheimer's disease, experimental evidence from post-mortem brain tissue suggest that the insoluble aggregates of tau are comprised of 3R and 4R tau. In rarer tauopathy diseases, such as progressive supranuclear palsy (PSP) and cortical basal degeneration (CBD), the 4R tau proteins are the aggregate-prone species of tau. The reasons underlying these differential types of aggregates in diseases is unknown. Therapies which target total tau (e.g., 3R+4R tau) versus only 4R tau could have beneficial effects in different disease states.

One major challenge, to date, has been developing assays to measure the mRNA of different isoforms of tau. For example, the “R/repeat” domain of tau makes it challenging to design primer pairs and TaqMan probes to measure 3R tau. Because of this paucity of assays for measuring certain isoforms of tau, it is challenging to accurately test the specificity of reagents (e.g., siRNAs) that should only reduce 4R tau and not affect 3R tau, versus reagents (e.g., siRNAs) that should reduce both (e.g., total tau targeting strategy).

We wanted to identify 4R-tau targeting siRNAs, for example, but the lack of mRNA assays to measure 3R tau made this type of screen impossible to perform. To circumvent this problem, we generated of a cellular screening assay to identify compounds (e.g., siRNA or gRNA) which target total tau versus compounds that specifically target 4R tau. This involved generating a secondary cell line (e.g., HEK293) that expressed fluorescently tagged fusion proteins of 4R and 3R tau. More specifically, the HEK293 cell line equally expressed both xN4R-tau-eYFP and xN3R-tau-mCherry (where xN=0N, 1N or 2N), providing a simple and robust system for screening compounds which specifically target 4R tau. Moreover, the fusion messenger RNA and protein can be used as surrogate readouts for the reduction of each form of tau. For example, an siRNA that targets 4R tau should decrease four readouts: (1) 4R tau mRNA using 4R-specific TaqMan probes; (2) eYFP mRNA using eYFP-specific TaqMan probes; (3) eYFP protein levels (e.g., measured via any protein detection technique (western blotting, ELISA)); and (4) eYFP fluorescence. Alternatively, an siRNA that also reduces 3R tau should impact: (1) mCherry messenger RNA using mCherry-specific TaqMan probes; (2) mCherry protein levels (e.g., measured via any protein detection technique (western blotting, ELISA)); and (3) mCherry fluorescence.

This cell line can be used for the screening of many different therapeutic modalities, including siRNAs, gRNAs, and intrabodies (intracellular antibodies). Intrabodies can be useful for the intracellular degradation of cytoplasmic-located proteins. For example, an intrabody which targets 4R tau or 3R tau should decrease yellow (eYFP) or red (mCherry) protein signals, respectively.

To make the cell line, full-length human 2N4R tau (NCBI Accession No: NM_005910.6), and 2N3R tau (NCBI Accession No: NM_001203252.2) were cloned into pLvX vector in-frame with either eYFP or mCherry, creating 2N4R-eYFP and 2N3R-mCherry fusion proteins. The fusion protein coding sequences were operably linked to an EF1A promoter. Plasmid DNA was packaged into lentiviruses. The 2N4R-eYFP fusion protein is set forth in SEQ ID NO: 15 and is encoded by the sequence set forth in SEQ ID NO: 19. The 2N3R-mCherry fusion protein is set forth in SEQ ID NO: 16 and is encoded by the sequence set forth in SEQ ID NO: 20.

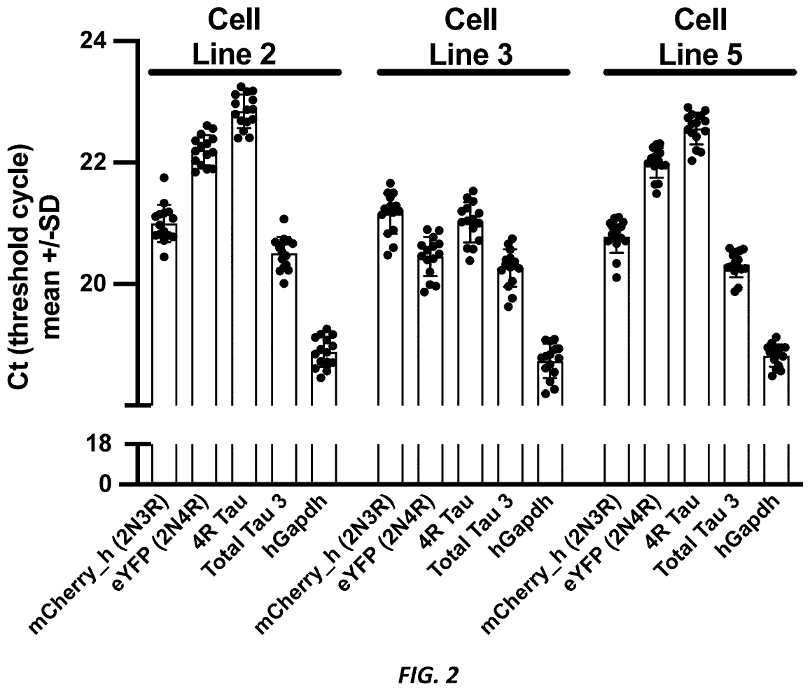

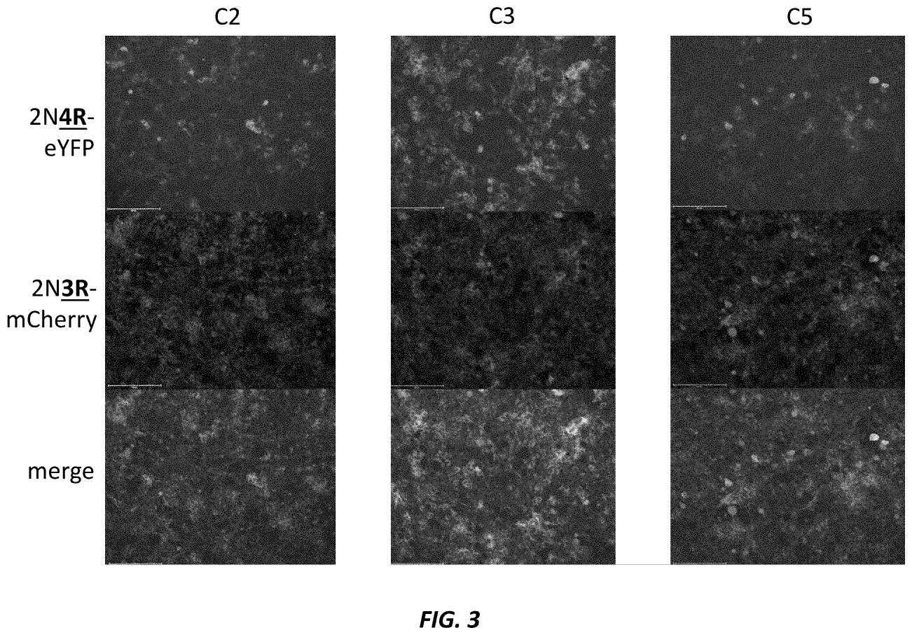

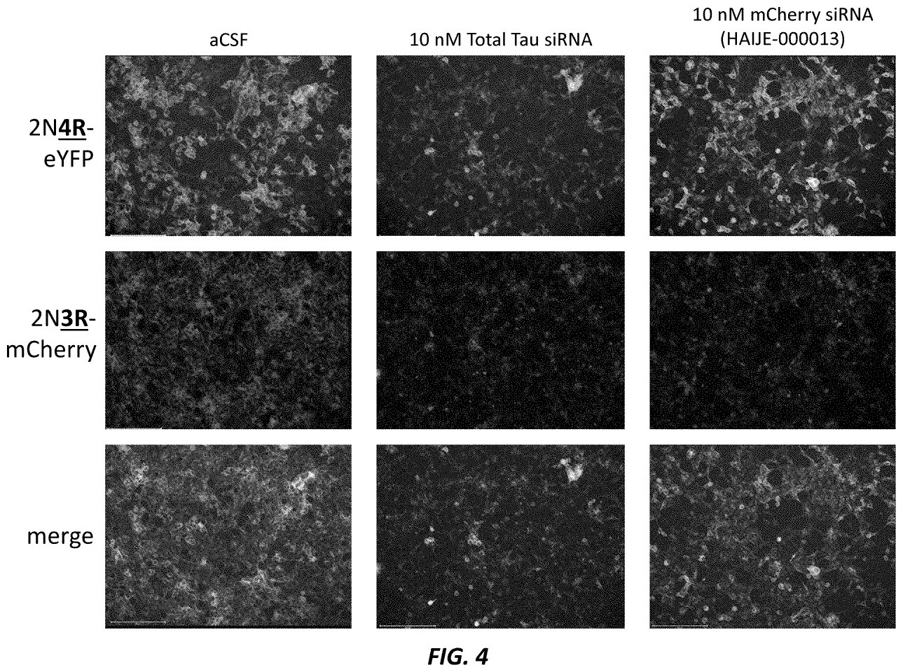

HEK293 cells were grown to 80% confluency in 24-well plates and grown in 10% fetal bovine serum in high-glucose DMEM. The cells were then transduced with the lentivirus carrying 2N4R-eYFP. After 3 days, eYFP+ cells were sorted into single cells in individual wells for expansion of clonal cell lines. Once eYFP+ cells reached confluency, five cells lines were chosen for subsequent cryopreservation and further expanded for transduction with lentivirus carrying 2N3R-mCherry. Following another 3 days, eYFP+ mCherry+ cells were sorted into single cells in 96-well plates and cells were grown for approximately two weeks to identify surviving clones that continued to proliferate. Twenty different eYFP/mCherry double-positive clonal cell lines were generated and used for subsequent screening in TaqMan assays. Three 2N4R-eYFP/2N3R-mCherry cell lines (clones C2, C3, and C5) expressed approximately 1:1 ratios of eYFP and mCherry mRNA. See FIG. 2. Clone C3 was selected as having high copy numbers of 2N4R-eYFP and 2N3R mCherry RNA and similar relative expression levels of each. See FIG. 3.

The HEK293, 2N4R-eYFP/2N3R-mCherry cells were maintained in complete medium (DMEM, 10% FBS, penicillin, streptomycin, GlutaMax, G418, and hygromycin) at 37° C., 5% CO₂. To show that eYFP and mCherry can serve as proxies for 4R tau and 3R tau levels, respectively, the dual reporter 2N4R-eYFP/2N3R-mCherry cells were plated in a 24-well plate at 1.6×10⁵ cells/well. On the day of transfection (˜70% confluence), the complete medium was aspirated from each well and was replaced with 450 uL OptiMEM. siRNA duplex solutions were diluted in OptiMem at 200 nM, 20 nM, and 2 nM. Lipofectamine RNAMax was diluted in OptiMem according to manufacturer's protocol (3 uL Lipofectamine/50 uL of media). Equal volumes of siRNA duplex solutions and diluted lipofectamine solutions were combined (10× siRNA/lipid solution), mixed, and incubated at room temperature for a minimum of 5 minutes. 50 uL of each 10×siRNA/lipid solution was added the appropriate well of a 24-well plate in triplicate. The plates were then returned to the incubator for 48 hours after which immunofluorescence analysis was done. Specifically, the cells were treated with either 10 nM total tau siRNA (targeting both 3R and 4R isoforms) or 10 nM mCherry siRNA (HAIJE-000013; SEQ ID NOS: 1 and 2). After 48 hours, eYFP and mCherry immunofluorescence was measured. The mCherry siRNA reduced the mCherry signal, but the eYFP signal was unaffected. In contrast, the total tau siRNA reduced both the eYFP signal intensity and the mCherry intensity. See FIG. 4.

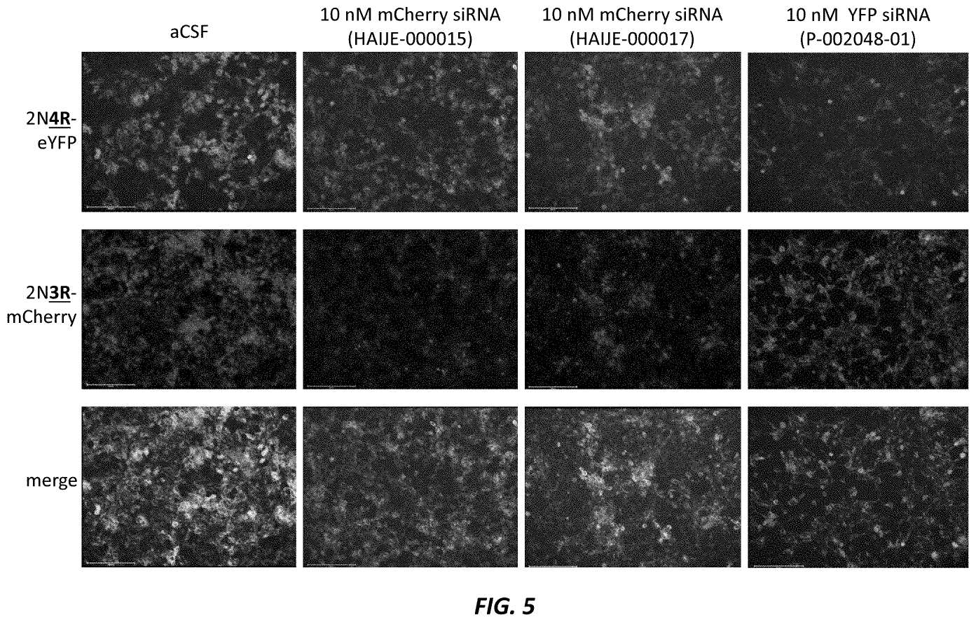

A similar experiment was then done using 10 nM mCherry siRNA (HAIJE-000015 (SEQ ID NOS: 3 and 4) or HAIJE-000017 (SEQ ID NOS: 5 and 6)) or 10 nM YFP siRNA (P-002048-01; SEQ ID NOS: 7 and 8). Both mCherry siRNAs reduced the mCherry signal intensity but not the eYFP signal intensity, whereas the YFP siRNA reduced the eYFP signal intensity but not the mCherry signal intensity. See FIG. 5.

Potential 4R-specific siRNAs were then designed by designing 65 siRNAs along exon 10 of the MAPT gene, which encodes the R2 domain. The R2 domain is set forth in SEQ ID NO: 21, and the coding sequence for the R2 domain is set forth in SEQ ID NO: 22. Two total tau siRNAs were designed as a positive control, the YFP siRNA was used as an eYFP knockdown control, and an mCherry siRNA was used as an mCherry knockdown control. Doses of 0.1 nM and 1 nM were tested in triplicate at 24 hours after treatment. TaqMan assays for total tau, 4R tau, mCherry, and eYFP were performed.

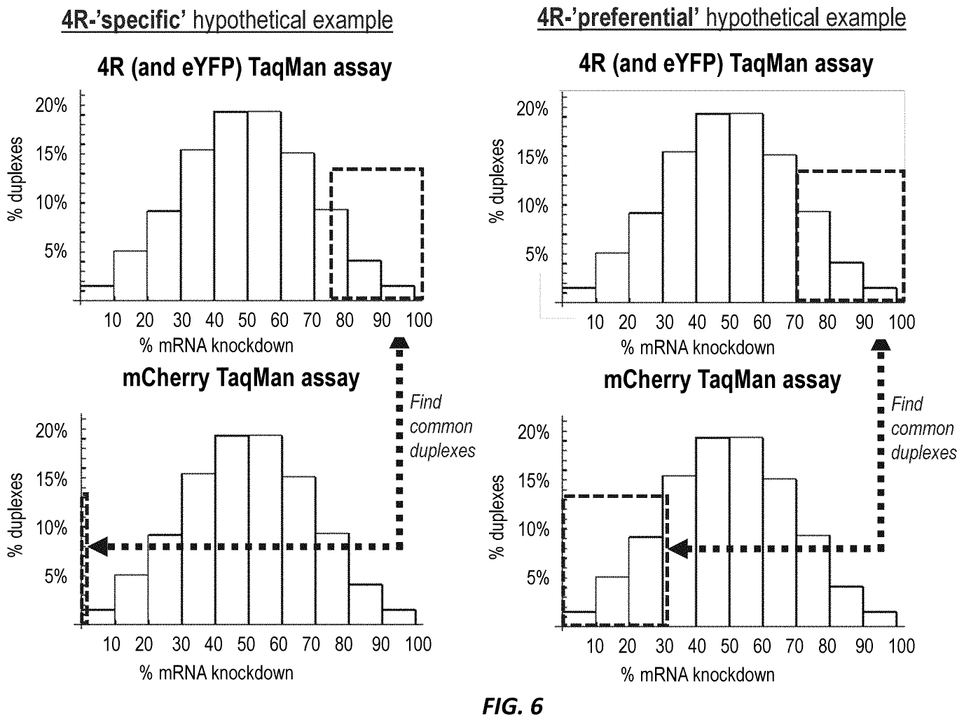

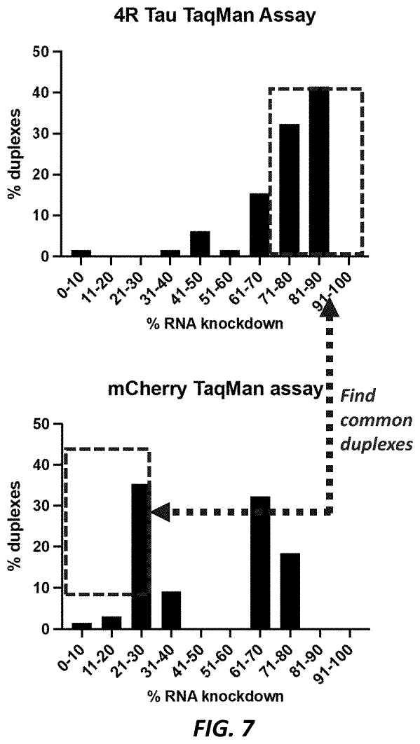

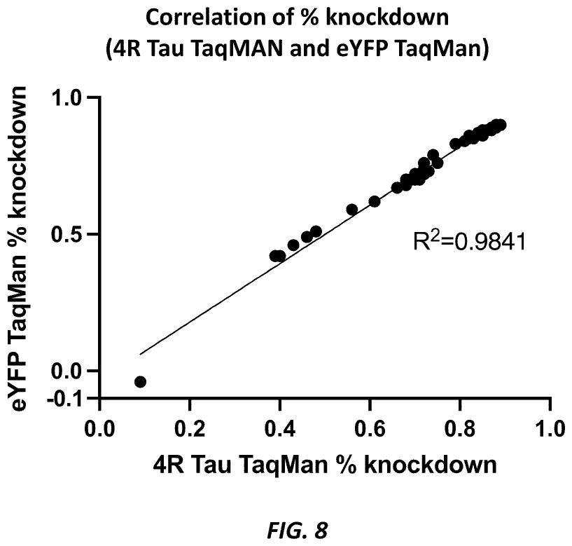

To select 4R-specific or 4R-preferential siRNAs, parameters can be set, for example, as in FIG. 6. A purely 4R-specific siRNA can be selected, for example, as one with robust knockdown of 4R tau and eYFP (e.g., at least 75% reduction of mRNA) and no effect on mCherry (e.g., −0% reduction of mCherry mRNA). A 4R-preferential siRNA can be selected, for example, as one with robust knockdown of 4R tau and eYFP (e.g., at least 70% reduction of mRNA) and little to no knockdown effect on mCherry (e.g., no more than 30% reduction of mCherry mRNA). Using the “4R-preferential” criteria, 13 of the 65 candidate 4R siRNAs met the criteria when dosed at 1 nM (no more than 30% of 4R tau message remaining and no greater than 30% knockdown of mCherry at 1 nM). See FIG. 7. We also found a near perfect correlation in the TaqMan assays for 4R tau and eYFP, validating the eYFP as a proxy for 4R tau levels. See FIG. 8. Similarly, mCherry was a reliable proxy for 3R tau levels.

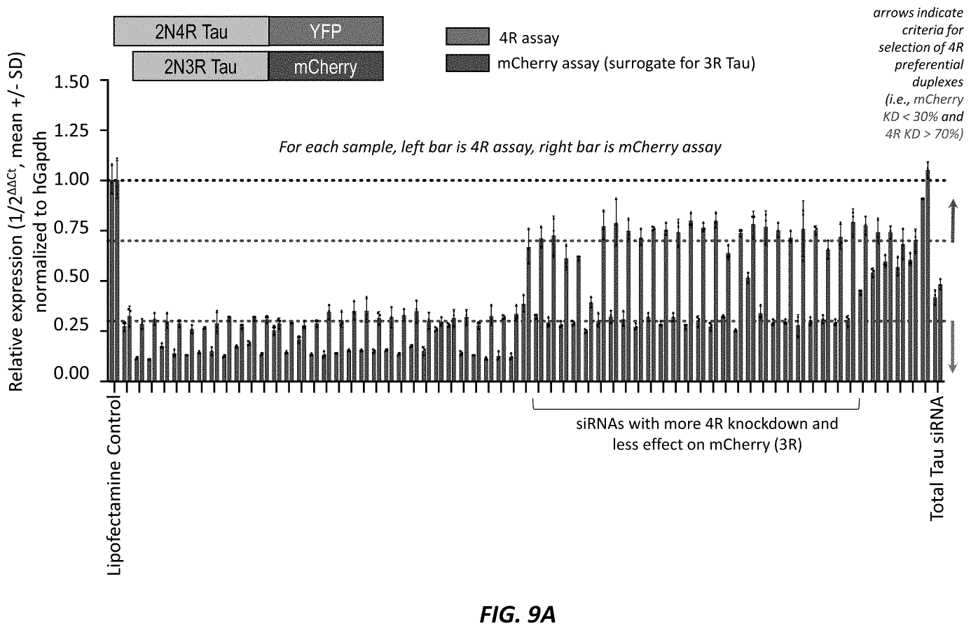

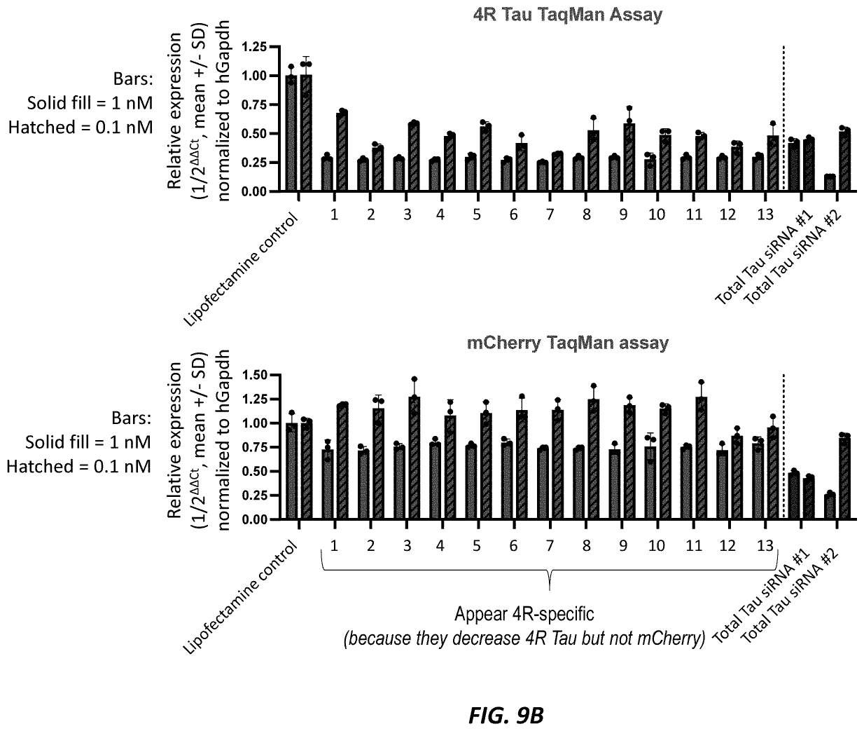

Relative 4R tau and mCherry expression for the 65 candidate 4R siRNA duplexes are shown in FIG. 9A. HEK293-2N4R-YFP+2N3R-mCherry cells were treated with 1 nM of GalNAc-conjugated siRNA in lipofectamine and harvested 48 hours later for TaqMan assays (mCherry and YFP). For each sample, the left bar shows the results of the 4R assay, and the right bar shows the results for the mCherry assay (surrogate for 3R tau). Relative 4R tau and mCherry expression for the 13 selected siRNAs are shown in FIG. 9B. The solid fill bars were samples treated with 1 nM siRNA, and the hatched bars were samples treated with 0.1 nM siRNAs. This validated the cell line as a tool for screening of 4R-specific reagents. Until now, screening of 4R-specific reagents such as siRNAs was impossible due to the lack of protein and mRNA assays to measure 3R tau. Addressing this problem, we created and validated a dual-reporter HEK293 2N4R-eYFP+2N3R-mCherry fusion protein cell line to screen for 4R-specific candidates.

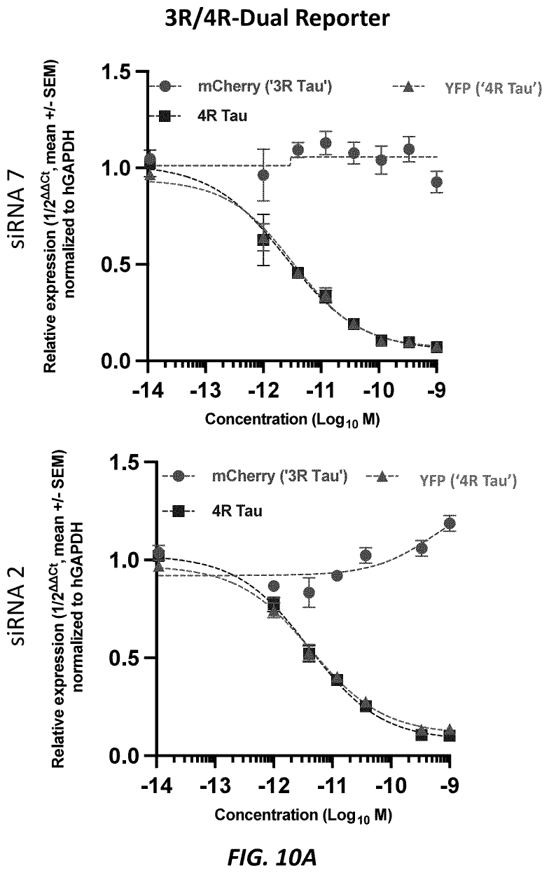

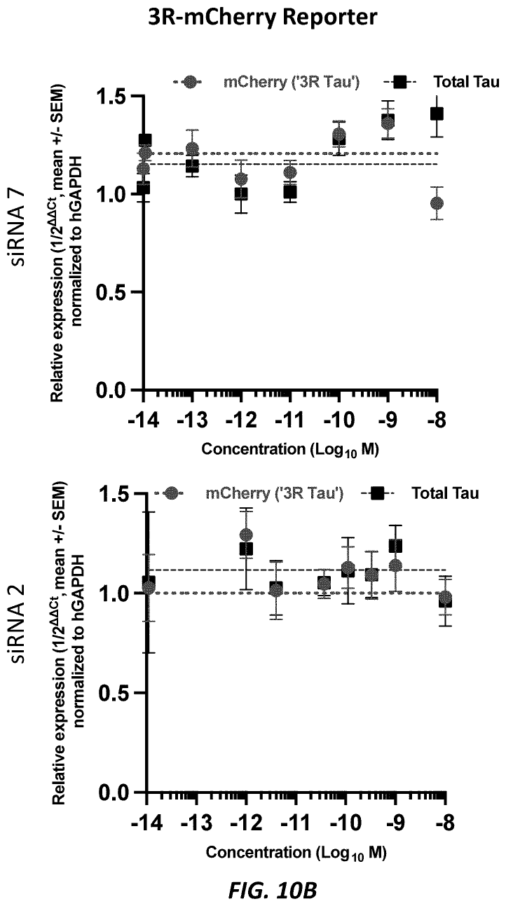

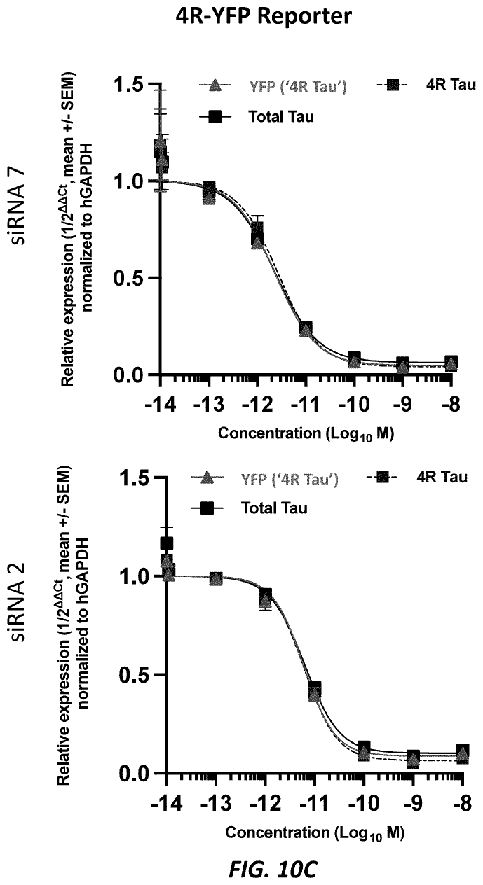

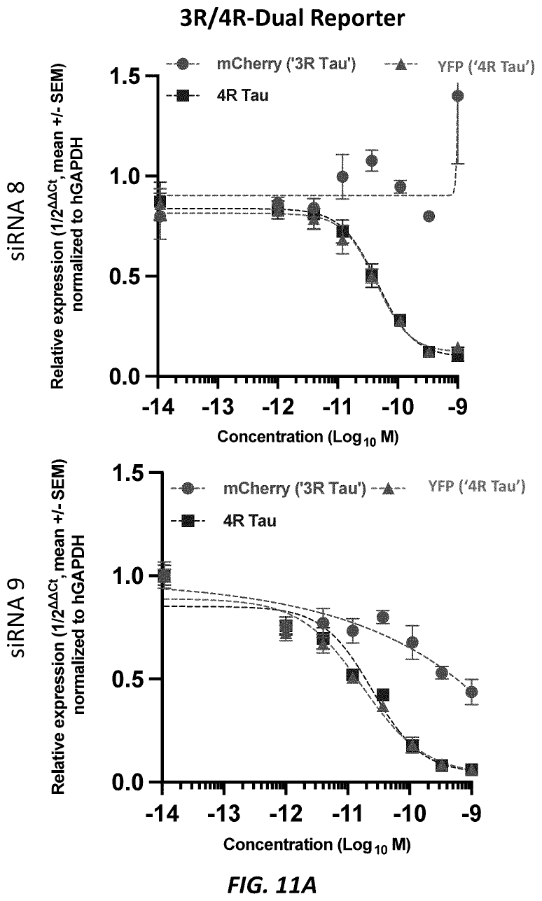

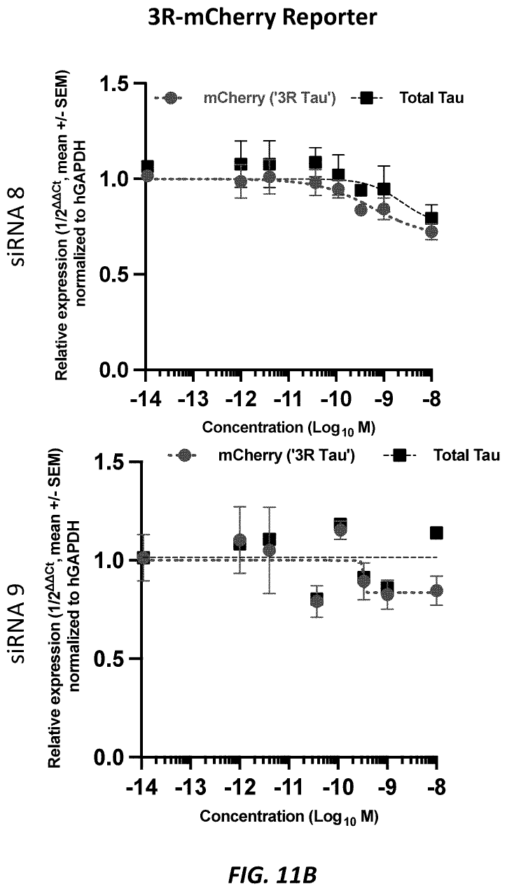

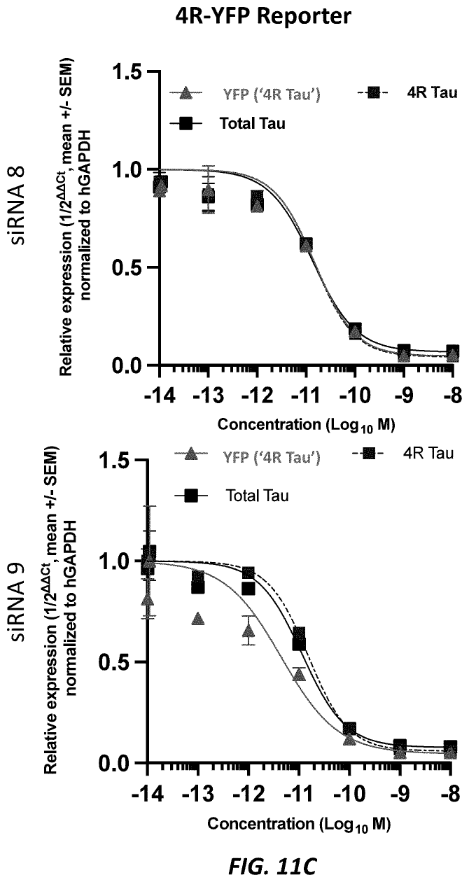

Dose-responses analyses were performed with each compound as above, using TaqMan to look at relative 4R tau expression and relative eYFP expression (4R tau surrogate) as well as relative mCherry expression (3R tau surrogate) using the 3R/4R-dual reporter cells. Results for two 4R tau-preferential siRNAs are shown in FIG. 10A, and results for two additional candidate 4R tau siRNAs are shown in FIG. 11A. The data show that the 4R preferential siRNAs are highly specific for 4R tau, with little to no-effect on 3R tau. However, based on sequence homology of the R2 domain to the other R domains, we wanted to ensure that the 4R preferential siRNAs retained the specificity if only given the 2N3R-mCherry target. Thus, we performed an identical set of experiments in HEK293 2N3R-mCherry only cells using the same dose ranges tested in the dual reporter cell line. We found no effect of the 4R siRNA molecules when tested in this cell system (i.e., no knockdown of 2N3R-mCherry). See FIGS. 10B and 11B. As a final control, we performed the same experiment in HEK293 2N4R-YFP only cells, where the potency of the 4R-preferential siRNAs was still observed. See FIGS. 10C and 11C.

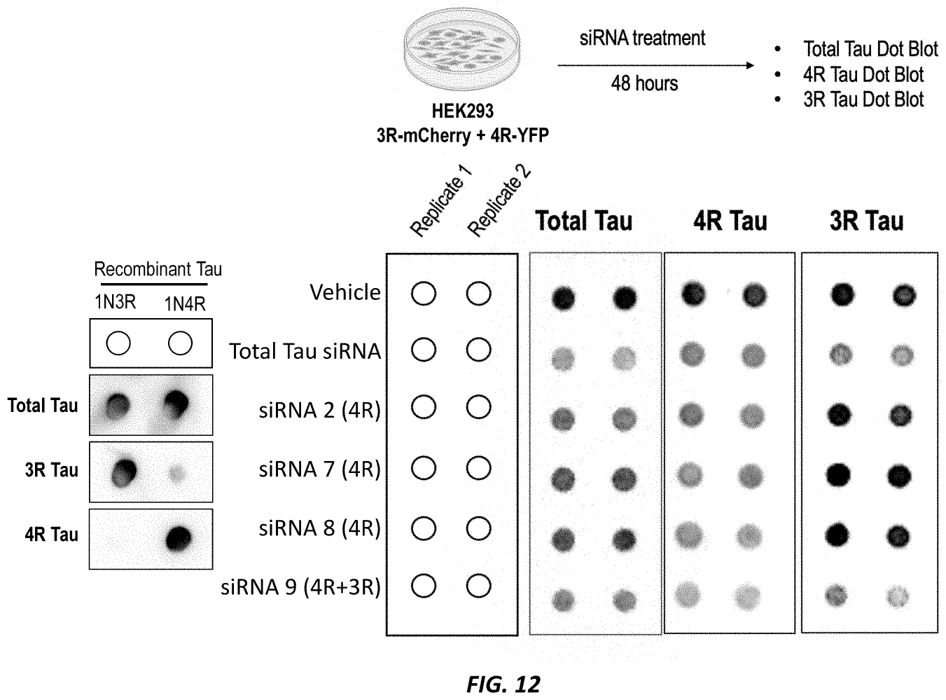

The same four 4R tau siRNAs were then validated for 4R tau protein knockdown using protein dot blotting with commercially available total tau antibody (mouse monoclonal; clone Tau12), 4R tau antibody (mouse monoclonal, clone 7D12.1), and 3R tau antibody (mouse monoclonal, clone 8E6\C11). Antibodies were first validated to confirm specificity by blotting with recombinant 1N3R or 1N4R proteins (left panel of FIG. 12). HEK293 dual reporter cells were then treated with vehicle control or siRNA (1 nM for 48 hours) in lipofectamine RNAiMax transfection reagent (diluted according to the manufacturer's instructions) (three 4R preferential siRNAs; and one mixed selectivity siRNA). Lysates were collected in RIPA buffer supplemented with protease/phosphatase inhibitors, a total of 7 μg (for total tau dot blot), or 21 μg (for 4R tau dot blot), or 7 μg (for 3R tau dot blot) was spotted into the dot blot apparatus, and blots were subjected to blotting with the total tau, 4R tau and 3R tau antibodies. The results are shown in FIG. 12 and further validate the dual-reporter HEK293 2N4R-eYFP+2N3R-mCherry fusion protein cell line to screen for 4R-specific candidates.

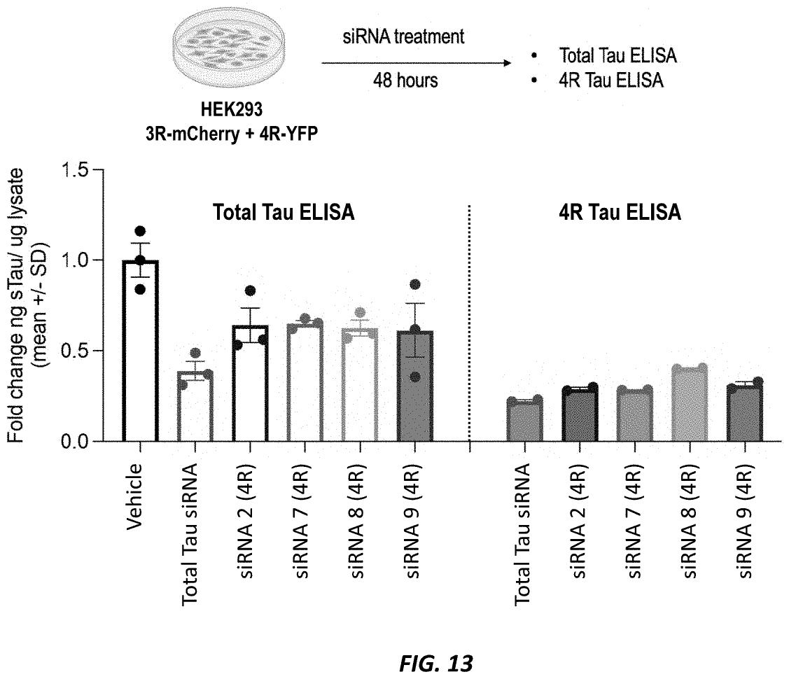

We also validated 4R preferential knockdown with the same 4R tau siRNAs using protein ELISA-type assays. As shown in FIG. 13, a commercially available total tau ALPHALISA (Perkin Elmer, cat no: AL271C) was used to measure total soluble tau according to the manufacturer's instructions, and 4R tau protein was measured using the PathScan ELISA kit from Cell Signaling Technologies (cat no: 29443). As predicted, 4R tau levels were lower than that of total tau, since the total tau assay is measuring levels both 4R tau (should be reduced) and 3R tau (should be minimally or not affected). A 3R tau ELISA assay is performed to quantitatively measure 3R tau levels. Flow cytometric analysis of eYFP and mCherry intensities is also done to assess effects on protein levels.

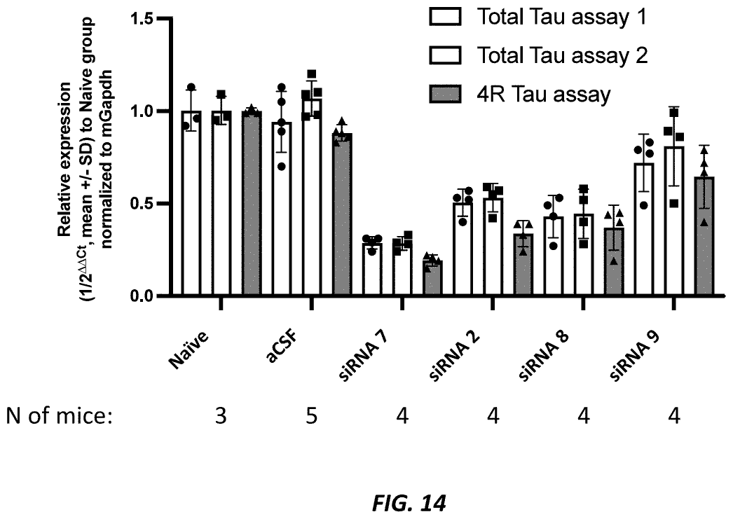

The same 4R tau siRNAs were then tested in vivo in MAPT humanized mice to further validate the dual-reporter HEK293 2N4R-eYFP+2N3R-mCherry fusion protein cell line. siRNAs with identical targeting sequences were synthesized with C16 conjugates to allow for CNS cell delivery. We then performed intracerebral injection of 300 μg of each siRNA and allowed the siRNA to incubate in vivo for 30 days. Naïve and vehicle (artificial cerebrospinal fluid (aCSF)) were used as negative controls. Total tau TaqMan assays (to assess global levels of 3R+4R tau) and 4R tau assays were performe, and the results are shown in FIG. 14.

Dot blotting was performed with the total tau, 3R tau, and 4R tau antibodies on the brain lysates of the MAPT humanized mice that were treated with the 4R tau siRNAs. Lysates were collected, and a total of 1 μg/well (for total tau dot blot), 1 μg/well (for 4R tau dot blot), or 5 μg/well (for 3R tau dot blot) was spotted into the dot blot apparatus, and blots were subjected to blotting with the total tau, 4R tau and 3R tau antibodies. The results confirmed the 4R preferential knockdown for the top 4 siRNAs (data not shown). RNAscope assays to detect mRNA for 3R tau, 4R tau, and total tau are performed on neuronal cultures (in vitro) and on slices from the brains of mice treated with the 4R tau preferential siRNAs.

### Example 2. Development of an In Vivo Screening Assay to Distinguish Reagents Targeting 4R Versus 3R Tau

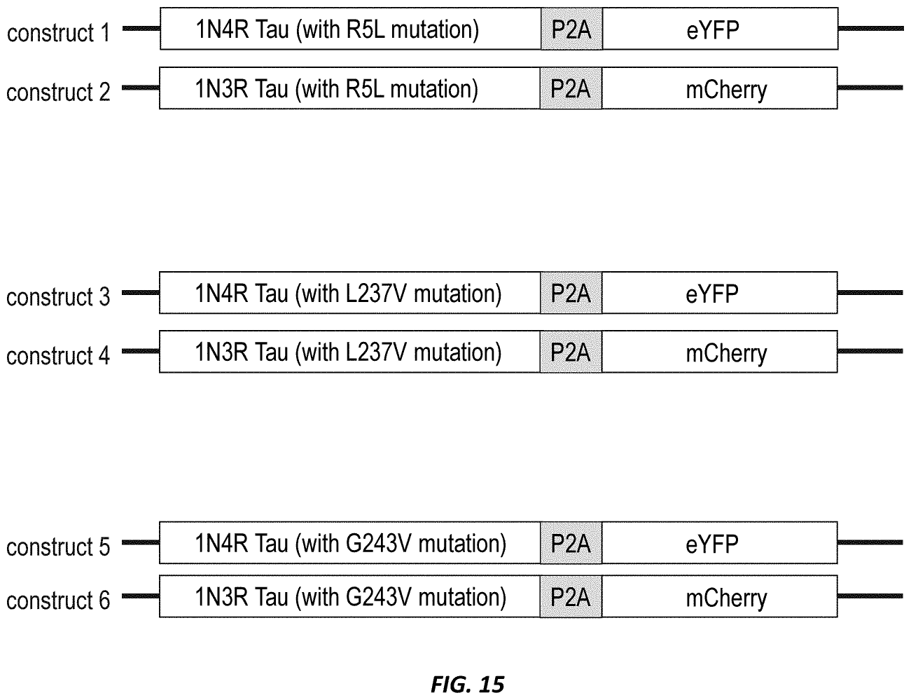

A mouse model for in vivo testing of 4R siRNAs is generated. Two constructs are virally introduced into tau knockout mice using AAV-PhPeB with a synapsin promoter (driving robust expression in neurons of the central nervous system): 1N4R tau(R5L)-P2A-eYFP and 1N3R tau(R5L)-P2A-mCherry. The 1N4R tau(R5L)-P2A-eYFP fusion protein is set forth in SEQ ID NO: 35 and is encoded by the sequence set forth in SEQ ID NO: 36. The 1N3R tau(R5L)-P2A-mCherry fusion protein is set forth in SEQ ID NO: 41 and is encoded by the sequence set forth in SEQ ID NO: 42. Similarly, two constructs are virally introduced into tau knockout mice using AAV-PhPeB with a synapsin promoter (driving robust expression in neurons of the central nervous system): 1N4R tau(L237V)-P2A-eYFP and 1N3R tau(L237V)-P2A-mCherry. The 1N4R tau(L237V)-P2A-eYFP fusion protein is set forth in SEQ ID NO: 37 and is encoded by the sequence set forth in SEQ ID NO: 38. The 1N3R tau(L237V)-P2A-mCherry fusion protein is set forth in SEQ ID NO: 43 and is encoded by the sequence set forth in SEQ ID NO: 44. Similarly, two constructs are virally introduced into tau knockout mice using AAV-PhPeB with a synapsin promoter (driving robust expression in neurons of the central nervous system): 1N4R tau(G243V)-P2A-eYFP and 1N3R tau(G243V)-P2A-mCherry. The 1N4R tau(G243V)-P2A-eYFP fusion protein is set forth in SEQ ID NO: 39 and is encoded by the sequence set forth in SEQ ID NO: 40. The 1N3R tau(G243V)-P2A-mCherry fusion protein is set forth in SEQ ID NO: 45 and is encoded by the sequence set forth in SEQ ID NO: 46. See FIG. 15. Similar to the in vitro screen with the cell line described above, eYFP and mCherry mRNA levels are used as proxy readouts for 4R tau and 3R tau mRNA levels, respectively. The difference is that, because we are driving neuronal expression of aggregate-prone tau, we do not want the eYFP and mCherry to interfere with proper function/folding and microtubule binding of these aggregate-prone tau forms. Thus, P2A is used in each construct, which causes two proteins to be produced from a single mRNA molecule due to ribosome skipping.

The mouse models are used to test candidate 4R tau targeting reagents, using readouts as described in Example 1. Behavioral metrics are performed to assess motor and ocular deficits. Behavior and disease pathology such as tau aggregates are assessed.

We claim:

1. A cell comprising a four-repeat (4R) tau isoform linked to a first reporter protein and a three-repeat (3R) tau isoform linked to a second reporter protein that is different from the first reporter protein.

2. The cell of claim 1, wherein the cell comprises a first fusion protein comprising the 4R tau isoform fused to the first reporter protein and a second fusion protein comprising the 3R tau isoform fused to the second reporter protein.

3. The cell of claim 2, wherein the cell comprises a first nucleic acid encoding the first fusion protein and a second nucleic acid encoding the second fusion protein, wherein the cell expresses the first fusion protein and the second fusion protein.

4. The cell of claim 1, wherein the cell comprises a first nucleic acid comprising a coding sequence for the 4R tau isoform and a coding sequence for the first reporter protein and a second nucleic acid comprising a coding sequence for the 3R tau isoform and a coding sequence for the second reporter protein, wherein the cell expresses the 4R tau isoform, the 3R tau isoform, the first reporter protein, and the second reporter protein.

5. The cell of claim 4, wherein the coding sequence for the 4R tau isoform and the coding sequence for the first reporter protein are separated by a coding sequence for a first 2A peptide, and the coding sequence for the 3R tau isoform and the coding sequence for the second reporter protein are separated by a coding sequence for a second 2A peptide.

6. The cell of claim 5, wherein the first 2A peptide is a first P2A peptide, and the second 2A peptide is a second P2A peptide.

7. The cell of any one of claims 3-6, wherein the first nucleic acid and the second nucleic acid are integrated into the genome of the cell.

8. The cell of any one of claims 3-7, wherein the cell comprises a viral vector comprising the first nucleic acid and the second nucleic acid.

9. The cell of claim 8, wherein the viral vector is a lentivirus vector or an adeno-associated virus (AAV) vector.

10. The cell of any one of claims 3-7, wherein the cell comprises a first viral vector comprising the first nucleic acid and a second viral vector comprising the second nucleic acid.

11. The cell of claim 10, wherein the first viral vector and the second viral vector are lentivirus vectors or adeno-associated virus (AAV) vectors.

12. The cell of any preceding claim, wherein the first reporter protein is a first fluorescent reporter protein, and the second reporter protein is a second fluorescent reporter protein.

13. The cell of any preceding claim, wherein the first reporter protein is eYFP, and the second reporter protein is mCherry.

14. The cell of any preceding claim, wherein the 4R tau isoform and the 3R tau isoform are human.

15. The cell of any preceding claim, wherein the 4R tau isoform is a 2N4R tau isoform.

16. The cell of any preceding claim, wherein the 3R tau isoform is a 2N3R tau isoform.

17. The cell of any one of claims 1-14, wherein the 4R tau isoform is a 2N4R tau isoform, and wherein the 3R tau isoform is a 2N3R tau isoform.

18. The cell of any preceding claim, wherein the 4R tau isoform comprises the sequence set forth in SEQ ID NO: 13, and the 3R tau isoform comprises the sequence set forth in SEQ ID NO: 14.

19. The cell of any one of claims 1-14, wherein the 4R tau isoform is a 1N4R tau isoform.

20. The cell of any one of claims 1-14 and 19, wherein the 3R tau isoform is a 1N3R tau isoform.

21. The cell of any one of claims 1-14, wherein the 4R tau isoform is a 1N4R tau isoform, and wherein the 3R tau isoform is a 1N3R tau isoform.

22. The cell of any one of claims 1-14 and 19-21, wherein the 4R tau isoform comprises the sequence set forth in SEQ ID NO: 23, 27, 31, or 47, and the 3R tau isoform comprises the sequence set forth in SEQ ID NO: 24, 28, 32, or 49.

23. The cell of any preceding claim, wherein the cell is a mammalian cell.

24. The cell of any preceding claim, wherein the cell is a human cell.

25. The cell of any preceding claim, wherein the cell is an immortalized cell.

26. The cell of any preceding claim, wherein the cell is a HEK293 cell.

27. A population of cells comprising a plurality of the cell of any preceding claim.

28. A non-human animal comprising a four-repeat (4R) tau isoform linked to a first reporter protein and a three-repeat (3R) tau isoform linked to a second reporter protein that is different from the first reporter protein.

29. The non-human animal of claim 28, wherein the non-human animal comprises a first fusion protein comprising the 4R tau isoform fused to the first reporter protein and a second fusion protein comprising the 3R tau isoform fused to the second reporter protein.

30. The non-human animal of claim 29, wherein the non-human animal comprises a first nucleic acid encoding the first fusion protein and a second nucleic acid encoding the second fusion protein, wherein the non-human animal expresses the first fusion protein and the second fusion protein.

31. The non-human animal of claim 28, wherein the non-human animal comprises a first nucleic acid comprising a coding sequence for the 4R tau isoform and a coding sequence for the first reporter protein and a second nucleic acid comprising a coding sequence for the 3R tau isoform and a coding sequence for the second reporter protein, wherein the non-human animal expresses the 4R tau isoform, the 3R tau isoform, the first reporter protein, and the second reporter protein.

32. The non-human animal of claim 31, wherein the coding sequence for the 4R tau isoform and the coding sequence for the first reporter protein are separated by a coding sequence for a first 2A peptide, and the coding sequence for the 3R tau isoform and the coding sequence for the second reporter protein are separated by a coding sequence for a second 2A peptide.

33. The non-human animal of claim 32, wherein the first 2A peptide is a first P2A peptide, and the second 2A peptide is a second P2A peptide.

34. The non-human animal of any one of claims 30-33, wherein the first nucleic acid and the second nucleic acid are integrated into the genome of the non-human animal.

35. The non-human animal of any one of claims 30-34, wherein the non-human animal comprises a viral vector comprising the first nucleic acid and the second nucleic acid.

36. The non-human animal of claim 35, wherein the viral vector is a lentivirus vector or an adeno-associated virus (AAV) vector.

37. The non-human animal of any one of claims 30-34, wherein the non-human animal comprises a first viral vector comprising the first nucleic acid and a second viral vector comprising the second nucleic acid.

38. The non-human animal of claim 37, wherein the first viral vector and the second viral vector are lentivirus vectors or adeno-associated virus (AAV) vectors.

39. The non-human animal of any one of claims 28-38, wherein the first reporter protein is a first fluorescent reporter protein, and the second reporter protein is a second fluorescent reporter protein.

40. The non-human animal of any one of claims 28-39, wherein the first reporter protein is eYFP, and the second reporter protein is mCherry.

41. The non-human animal of any one of claims 28-40, wherein the 4R tau isoform and the 3R tau isoform are human, optionally wherein the 4R tau isoform and the 3R tau isoform each comprise a R5L mutation, a L237V mutation, or a G243V mutation, or optionally wherein the 4R tau isoform and the 3R tau isoform each comprise a N279K mutation, a L284R mutation, or a S285R mutation.

42. The non-human animal of any one of claims 28-41, wherein the 4R tau isoform is a 2N4R tau isoform.

43. The non-human animal of any one of claims 28-42, wherein the 3R tau isoform is a 2N3R tau isoform.

44. The non-human animal of any one of claims 28-41, wherein the 4R tau isoform is a 2N4R tau isoform, and wherein the 3R tau isoform is a 2N3R tau isoform.

45. The non-human animal of any one of claims 28-44, wherein the 4R tau isoform comprises the sequence set forth in SEQ ID NO: 13, and the 3R tau isoform comprises the sequence set forth in SEQ ID NO: 14.

46. The non-human animal of any one of claims 28-41, wherein the 4R tau isoform is a 1N4R tau isoform.

47. The non-human animal of any one of claims 28-41 and 46, wherein the 3R tau isoform is a 1N3R tau isoform.

48. The non-human animal of any one of claims 28-41, wherein the 4R tau isoform is a 1N4R tau isoform, and wherein the 3R tau isoform is a 1N3R tau isoform.

49. The non-human animal of any one of claims 28-41 and 46-48, wherein the 4R tau isoform comprises the sequence set forth in SEQ ID NO: 23, 27, 31, or 47, and the 3R tau isoform comprises the sequence set forth in SEQ ID NO: 24, 28, 32, or 49.

50. The non-human animal of any one of claims 28-49, wherein the non-human animal is a mammal.

51. The non-human animal of any one of claims 28-50, wherein the non-human animal is a rodent.

52. The non-human animal of any one of claims 28-51, wherein the non-human animal is a mouse.

53. The non-human animal of any one of claims 28-51, wherein the non-human animal is a rat.

54. The non-human animal of any one of claims 28-53, wherein the 4R tau isoform, the first reporter protein, the 3R tau isoform, and the second reporter protein are expressed in neurons of the central nervous system of the non-human animal.

55. The non-human animal of any one of claims 28-54, wherein the non-human animal comprises filamentous tau inclusions.

56. A method of assessing the activity of a tau-targeting reagent, comprising:
(a) administering the tau-targeting reagent to cell of any one of claims 1-26; and
(b) assessing the activity of the tau-targeting reagent in the cell.

57. The method of claim 56, wherein the activity of the tau-targeting reagent is assessed compared to a control cell that is not administered the tau-targeting reagent or is assessed compared to prior to administering the tau-targeting reagent.

58. The method of claim 56 or 57, wherein the assessing comprises measuring one or more of 4R tau messenger RNA expression, first reporter protein messenger RNA expression, and second reporter protein messenger RNA expression.

59. The method of any one of claims 56-58, wherein the assessing comprises measuring 4R tau isoform messenger RNA expression and second reporter protein messenger RNA expression,
wherein a larger relative decrease in 4R tau isoform messenger RNA expression compared to second reporter protein messenger RNA expression after administering the tau-targeting reagent to the cell indicates that the tau-targeting reagent is a 4R-preferential tau targeting reagent, optionally wherein a decrease of at least 70% in 4R tau isoform messenger RNA expression and a decrease of no more than 30% in second reporter protein messenger RNA expression after administering the tau-targeting reagent to the cell indicates that the tau-targeting reagent is a 4R-preferential tau targeting reagent.

60. The method of any one of claims 56-59, wherein the assessing comprises measuring first reporter protein messenger RNA expression and second reporter protein messenger RNA expression,
wherein a larger relative decrease in first reporter protein messenger RNA expression compared to second reporter protein messenger RNA expression after administering the tau-targeting reagent to the cell indicates that the tau-targeting reagent is a 4R-preferential tau targeting reagent, optionally wherein a decrease of at least 70% in first reporter protein messenger RNA expression and a decrease of no more than 30% in second reporter protein messenger RNA expression after administering the tau-targeting reagent to the cell indicates that the tau-targeting reagent is a 4R-preferential tau targeting reagent.

61. The method of any one of claims 56-60, wherein the assessing comprises measuring one or more of first reporter protein expression and second reporter protein expression.

62. The method of any one of claims 56-61, wherein the assessing comprises measuring first reporter protein expression and second reporter protein expression, wherein a larger relative decrease in first reporter protein expression compared to second reporter protein expression after administering the tau-targeting reagent to the cell indicates that the tau-targeting reagent is a 4R-preferential tau targeting reagent, optionally wherein a decrease of at least 70% in first reporter protein expression and a decrease of no more than 30% in second reporter protein expression after administering the tau-targeting reagent to the cell indicates that the tau-targeting reagent is a 4R-preferential tau targeting reagent.

63. The method of any one of claims 56-62, wherein the first reporter protein is a first fluorescent reporter protein, and the second reporter protein is a second fluorescent reporter protein, and the assessing in step (b) comprises immunofluorescence staining or flow cytometry.

64. The method of any one of claims 56-63, wherein the assessing in step (b) comprises assessing tau hyperphosphorylation or tau aggregation.

65. The method of any one of claims 56-64, wherein the tau-targeting reagent is an RNAi agent or an antisense oligonucleotide.

66. The method of any one of claims 56-64, wherein the tau-targeting reagent is an intrabody.

67. The method of any one of claims 56-64, wherein the tau-targeting reagent is a nuclease agent.

68. The method of claim 67, wherein the nuclease agent comprises a Cas protein and a guide RNA designed to target a guide RNA target sequence in a tau coding sequence.

69. A method of assessing the activity of a tau-targeting reagent in vivo, comprising:
(a) administering the tau-targeting reagent to the non-human animal of any one of claims 28-55; and
(b) assessing the activity of the tau-targeting reagent in the non-human animal.

70. The method of claim 69, wherein the activity of the tau-targeting reagent is assessed compared to a control non-human animal that is not administered the tau-targeting reagent or is assessed compared to prior to administering the tau-targeting reagent.

71. The method of 69 or 70, wherein the assessing comprises measuring one or more of 4R tau messenger RNA expression, first reporter protein messenger RNA expression, and second reporter protein messenger RNA expression.

72. The method of any one of claims 69-71, wherein the assessing comprises measuring 4R tau isoform messenger RNA expression and second reporter protein messenger RNA expression,
wherein a larger relative decrease in 4R tau isoform messenger RNA expression compared to second reporter protein messenger RNA expression after administering the tau-targeting reagent to the non-human animal indicates that the tau-targeting reagent is a 4R-preferential tau targeting reagent, optionally wherein a decrease of at least 70% in 4R tau isoform messenger RNA expression and a decrease of no more than 30% in second reporter protein messenger RNA expression after administering the tau-targeting reagent to the non-human animal indicates that the tau-targeting reagent is a 4R-preferential tau targeting reagent.

73. The method of any one of claims 69-72, wherein the assessing comprises measuring first reporter protein messenger RNA expression and second reporter protein messenger RNA expression,
wherein a larger relative decrease in first reporter protein messenger RNA expression compared to second reporter protein messenger RNA expression after administering the tau-targeting reagent to the non-human animal indicates that the tau-targeting reagent is a 4R-preferential tau targeting reagent, optionally wherein a decrease of at least 70% in first reporter protein messenger RNA expression and a decrease of no more than 30% in second reporter protein messenger RNA expression after administering the tau-targeting reagent to the non-human animal indicates that the tau-targeting reagent is a 4R-preferential tau targeting reagent.

74. The method of any one of claims 69-73, wherein the assessing comprises measuring one or more of first reporter protein expression and second reporter protein expression.

75. The method of any one of claims 69-74, wherein the assessing comprises measuring first reporter protein expression and second reporter protein expression,
wherein a larger relative decrease in first reporter protein expression compared to second reporter protein expression after administering the tau-targeting reagent to the non-human animal indicates that the tau-targeting reagent is a 4R-preferential tau targeting reagent, optionally wherein a decrease of at least 70% in first reporter protein expression and a decrease of no more than 30% in second reporter protein expression after administering the tau-targeting reagent to the non-human animal indicates that the tau-targeting reagent is a 4R-preferential tau targeting reagent.

76. The method of any one of claims 69-75, wherein the first reporter protein is a first fluorescent reporter protein, and the second reporter protein is a second fluorescent reporter protein, and the assessing in step (b) comprises immunofluorescence staining or flow cytometry.

77. The method of any one of claims 69-76, wherein the assessing in step (b) comprises assessing tau hyperphosphorylation or tau aggregation.

78. The method of any one of claims 69-77, wherein the tau-targeting reagent is an RNAi agent or an antisense oligonucleotide.

79. The method of any one of claims 69-77, wherein the tau-targeting reagent is an intrabody.

80. The method of any one of claims 69-77, wherein the tau-targeting reagent is a nuclease agent.

81. The method of claim 80, wherein the nuclease agent comprises a Cas protein and a guide RNA designed to target a guide RNA target sequence in a tau coding sequence.

82. The method of any one of claims 69-81, wherein the assessing is in neurons in the central nervous system of the non-human animal.

83. A composition comprising:
(a) a four-repeat (4R) tau isoform linked to a first reporter protein and a three-repeat (3R) tau isoform linked to a second reporter protein that is different from the first reporter protein; or
(b) a first nucleic acid encoding the 4R tau isoform linked to the first reporter protein and a second nucleic acid encoding the 3R tau isoform linked to the second reporter protein.

84. The composition of claim 83, wherein the composition comprises a first fusion protein comprising the 4R tau isoform fused to the first reporter protein and a second fusion protein comprising the 3R tau isoform fused to the second reporter protein, or wherein the first nucleic acid encodes the first fusion protein and the second nucleic acid encodes the second fusion protein.

85. The composition of claim 83, wherein the first nucleic acid comprises a coding sequence for the 4R tau isoform and a coding sequence for the first reporter protein separated by a coding sequence for a first 2A peptide, and wherein the second nucleic acid comprises a coding sequence for the 3R tau isoform and a coding sequence for the second reporter protein separated by a coding sequence for a second 2A peptide.

86. The composition of claim 85, wherein the first 2A peptide is a first P2A peptide, and the second 2A peptide is a second P2A peptide.

87. The composition of any one of claims 83-86, wherein the first nucleic acid and the second nucleic acid are in a viral vector.

88. The composition of claim 87, wherein the viral vector is a lentivirus vector or an adeno-associated virus (AAV) vector.

89. The composition of any one of claims 83-86, wherein the first nucleic acid is in a first viral vector, and the second nucleic acid is in a second viral vector.

90. The composition of claim 89, wherein the first viral vector and the second viral vector are lentivirus vectors or adeno-associated virus (AAV) vectors.

91. The composition of any one of claims 83-90, wherein the first reporter protein is a first fluorescent reporter protein, and the second reporter protein is a second fluorescent reporter protein.

92. The composition of any one of claims 83-91, wherein the first reporter protein is eYFP, and the second reporter protein is mCherry.

93. The composition of any one of claims 83-92, wherein the 4R tau isoform and the 3R tau isoform are human.

94. The composition of any one of claims 83-93, wherein the 4R tau isoform is a 2N4R tau isoform.

95. The composition of any one of claims 83-94, wherein the 3R tau isoform is a 2N3R tau isoform.

96. The composition of any one of claims 83-93, wherein the 4R tau isoform is a 2N4R tau isoform, and wherein the 3R tau isoform is a 2N3R tau isoform.

97. The composition of any one of claims 83-96, wherein the 4R tau isoform comprises the sequence set forth in SEQ ID NO: 13, and the 3R tau isoform comprises the sequence set forth in SEQ ID NO: 14.

98. The composition of any one of claims 83-93, wherein the 4R tau isoform is a 1N4R tau isoform.

99. The composition of any one of claims 83-93 and 98, wherein the 3R tau isoform is a 1N3R tau isoform.

100. The composition of any one of claims 83-93, wherein the 4R tau isoform is a 1N4R tau isoform, and wherein the 3R tau isoform is a 1N3R tau isoform.

101. The composition of any one of claims 83-93 and 98-100, wherein the 4R tau isoform comprises the sequence set forth in SEQ ID NO: 23, 27, 31, or 47, and the 3R tau isoform comprises the sequence set forth in SEQ ID NO: 24, 28, 32, or 49.

102. A cell comprising the composition of any one of claims 83-101.

103. A non-human animal comprising the composition of any one of claims 83-101.

104. A method of making the cell of any one of claims 1-26 and 102, comprising introducing into the cell the four-repeat (4R) tau isoform linked to the first reporter protein and the three-repeat (3R) tau isoform linked to the second reporter protein, or introducing into the cell a first nucleic acid encoding the 4R tau isoform linked to the first reporter protein and a second nucleic acid encoding the 3R tau isoform linked to the second reporter protein.

105. A method of making the non-human animal of any one of claims 28-55 and 103, comprising administering to the non-human animal the four-repeat (4R) tau isoform linked to the first reporter protein and the three-repeat (3R) tau isoform linked to the second reporter protein, or administering to the non-human animal a first nucleic acid encoding the 4R tau isoform linked to the first reporter protein and a second nucleic acid encoding the 3R tau isoform linked to the second reporter protein.


SEQUENCE LISTING (50 sequences)

SEQ ID NO: 1 - RNA, 21 nt, synthetic construct
  misc_feature 1..21: note = Synthetic
  source 1..21: mol_type = other RNA
gcaagggcgaggaggataatt

SEQ ID NO: 2 - RNA, 21 nt, synthetic construct
  misc_feature 1..21: note = Synthetic
  source 1..21: mol_type = other RNA
ttatcctcctcgcccttgctt

SEQ ID NO: 3 - RNA, 21 nt, synthetic construct
  misc_feature 1..21: note = Synthetic
  source 1..21: mol_type = other RNA
cctacaacgtcaacatcaatt

SEQ ID NO: 4 - RNA, 21 nt, synthetic construct
  misc_feature 1..21: note = Synthetic
  source 1..21: mol_type = other RNA
ttgatgttgacgttgtaggtt

SEQ ID NO: 5 - RNA, 21 nt, synthetic construct
  misc_feature 1..21: note = Synthetic
  source 1..21: mol_type = other RNA
tggacgagctgtacaagtatt

SEQ ID NO: 6 - RNA, 21 nt, synthetic construct
  misc_feature 1..21: note = Synthetic
  source 1..21: mol_type = other RNA
tacttgtacagctcgtccatt

SEQ ID NO: 7 - RNA, 24 nt, synthetic construct
  misc_feature 1..24: note = Synthetic
  source 1..24: mol_type = other RNA
ttgcaagctgaccctgaagttctt

SEQ ID NO: 8 - RNA, 24 nt, synthetic construct
  misc_feature 1..24: note = Synthetic
  source 1..24: mol_type = other RNA
ttgaacttcagggtcagcttgctt

SEQ ID NO: 9 - protein, 18 aa, synthetic construct
  REGION 1..18: note = Synthetic
EGRGSLLTCGDVEENPGP

SEQ ID NO: 10 - protein, 19 aa, synthetic construct
  REGION 1..19: note = Synthetic
ATNFSLLKQAGDVEENPGP

SEQ ID NO: 11 - protein, 20 aa, synthetic construct
  REGION 1..20: note = Synthetic
QCTNYALLKLAGDVESNPGP

SEQ ID NO: 12 - protein, 22 aa, synthetic construct
  REGION 1..22: note = Synthetic
VKQTLNFDLLKLAGDVESNPGP

SEQ ID NO: 13 - protein, 441 aa, synthetic construct
  REGION 1..441: note = Synthetic
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 14 - protein, 410 aa, synthetic construct
  REGION 1..410: note = Synthetic
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 15 - protein, 680 aa, synthetic construct
  REGION 1..680: note = Synthetic
  REGION 1..441: note = MISC_FEATURE - 2N4R Tau
  REGION 442..680: note = MISC_FEATURE - eYFP
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 16 - protein, 646 aa, synthetic construct
  REGION 1..646: note = Synthetic
  REGION 1..410: note = MISC_FEATURE - 2N3R Tau
  REGION 411..646: note = MISC_FEATURE - mCherry
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 17 - DNA, 1323 bp, synthetic construct
  misc_feature 1..1323: note = Synthetic
  source 1..1323: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 18 - DNA, 1230 bp, synthetic construct
  misc_feature 1..1230: note = Synthetic
  source 1..1230: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 19 - DNA, 2040 bp, synthetic construct
  misc_feature 1..2040: note = Synthetic
  misc_feature 1..1323: note = 2N4R Tau
  misc_feature 1324..2040: note = eYFP
  source 1..2040: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaag

SEQ ID NO: 20 - DNA, 1938 bp, synthetic construct
  misc_feature 1..1938: note = Synthetic
  misc_feature 1..1230: note = 2N3R Tau
  misc_feature 1231..1938: note = mCherry
  source 1..1938: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 21 - protein, 31 aa, synthetic construct
  REGION 1..31: note = Synthetic
KVQIINKKLDLSNVQSKCGSKDNIKHVPGGG

SEQ ID NO: 22 - DNA, 93 bp, synthetic construct
  misc_feature 1..93: note = Synthetic
  source 1..93: mol_type = other DNA
aaggtgcagataattaataagaagctggatcttagcaacgtccagtccaagtgtggctcaaaggataatatcaaacacgtcccgggaggcggc

SEQ ID NO: 23 - protein, 412 aa, synthetic construct
  REGION 1..412: note = Synthetic
MAEPLQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 24 - protein, 381 aa, synthetic construct
  REGION 1..381: note = Synthetic
MAEPLQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 25 - DNA, 1236 bp, synthetic construct
  misc_feature 1..1236: note = Synthetic
  source 1..1236: mol_type = other DNA
atggctgagcccctccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 26 - DNA, 1143 bp, synthetic construct
  misc_feature 1..1143: note = Synthetic
  source 1..1143: mol_type = other DNA
atggctgagcccctccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 27 - protein, 412 aa, synthetic construct
  REGION 1..412: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENVKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 28 - protein, 381 aa, synthetic construct
  REGION 1..381: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENVKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 29 - DNA, 1236 bp, synthetic construct
  misc_feature 1..1236: note = Synthetic
  source 1..1236: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacgtgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 30 - DNA, 1143 bp, synthetic construct
  misc_feature 1..1143: note = Synthetic
  source 1..1143: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacgtgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 31 - protein, 412 aa, synthetic construct
  REGION 1..412: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGVGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 32 - protein, 381 aa, synthetic construct
  REGION 1..381: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGVGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 33 - DNA, 1236 bp, synthetic construct
  misc_feature 1..1236: note = Synthetic
  source 1..1236: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggagtcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 34 - DNA, 1143 bp, synthetic construct
  misc_feature 1..1143: note = Synthetic
  source 1..1143: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggagtcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 35 - protein, 680 aa, synthetic construct
  REGION 1..680: note = Synthetic
  REGION 1..412: note = MISC_FEATURE - 1N4R Tau
  SITE 5: note = MISC_FEATURE - Mutation
  REGION 413..419: note = MISC_FEATURE - Furin-GSG linker
  REGION 420..441: note = MISC_FEATURE - P2A
  REGION 442..680: note = MISC_FEATURE - eYFP
MAEPLQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 36 - DNA, 2040 bp, synthetic construct
  misc_feature 1..2040: note = Synthetic
  misc_feature 1..1236: note = 1N4R Tau
  misc_feature 13..15: note = Mutation
  misc_feature 1237..1257: note = Furin-GSG linker
  misc_feature 1258..1323: note = P2A
  misc_feature 1324..2040: note = eYFP
  source 1..2040: mol_type = other DNA
atggctgagcccctccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaag

SEQ ID NO: 37 - protein, 680 aa, synthetic construct
  REGION 1..680: note = Synthetic
  REGION 1..412: note = MISC_FEATURE - 1N4R Tau
  SITE 237: note = MISC_FEATURE - Mutation
  REGION 413..419: note = MISC_FEATURE - Furin-GSG linker
  REGION 420..441: note = MISC_FEATURE - P2A
  REGION 442..680: note = MISC_FEATURE - eYFP
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENVKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 38 - DNA, 2040 bp, synthetic construct
  misc_feature 1..2040: note = Synthetic
  misc_feature 1..1236: note = 1N4R Tau
  misc_feature 709..711: note = Mutation
  misc_feature 1237..1257: note = Furin-GSG linker
  misc_feature 1258..1323: note = P2A
  misc_feature 1324..2040: note = eYFP
  source 1..2040: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacgtgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaag

SEQ ID NO: 39 - protein, 680 aa, synthetic construct
  REGION 1..680: note = Synthetic
  REGION 1..412: note = MISC_FEATURE - 1N4R Tau
  SITE 243: note = MISC_FEATURE - Mutation
  REGION 413..419: note = MISC_FEATURE - Furin-GSG linker
  REGION 420..441: note = MISC_FEATURE - P2A
  REGION 442..680: note = MISC_FEATURE - eYFP
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGVGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 40 - DNA, 2040 bp, synthetic construct
  misc_feature 1..2040: note = Synthetic
  misc_feature 1..1236: note = 1N4R Tau
  misc_feature 727..729: note = Mutation
  misc_feature 1237..1257: note = Furin-GSG linker
  misc_feature 1258..1323: note = P2A
  misc_feature 1324..2040: note = eYFP
  source 1..2040: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggagtcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaag

SEQ ID NO: 41 - protein, 646 aa, synthetic construct
  REGION 1..646: note = Synthetic
  REGION 1..381: note = MISC_FEATURE - 1N3R Tau
  SITE 5: note = MISC_FEATURE - Mutation
  REGION 382..388: note = MISC_FEATURE - Furin-GSG linker
  REGION 389..410: note = MISC_FEATURE - P2A
  REGION 411..646: note = MISC_FEATURE - mCherry
MAEPLQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 42 - DNA, 1938 bp, synthetic construct
  misc_feature 1..1938: note = Synthetic
  misc_feature 1..1143: note = 1N3R Tau
  misc_feature 13..15: note = Mutation
  misc_feature 1144..1164: note = Furin-GSG linker
  misc_feature 1165..1230: note = P2A
  misc_feature 1231..1938: note = mCherry
  source 1..1938: mol_type = other DNA
atggctgagcccctccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 43 - protein, 646 aa, synthetic construct
  REGION 1..646: note = Synthetic
  REGION 1..381: note = MISC_FEATURE - 1N3R Tau
  SITE 237: note = MISC_FEATURE - Mutation
  REGION 382..388: note = MISC_FEATURE - Furin-GSG linker
  REGION 389..410: note = MISC_FEATURE - P2A
  REGION 411..646: note = MISC_FEATURE - mCherry
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENVKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 44 - DNA, 1938 bp, synthetic construct
  misc_feature 1..1938: note = Synthetic
  misc_feature 1..1143: note = 1N3R Tau
  misc_feature 709..711: note = Mutation
  misc_feature 1144..1164: note = Furin-GSG linker
  misc_feature 1165..1230: note = P2A
  misc_feature 1231..1938: note = mCherry
  source 1..1938: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacgtgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 45 - protein, 646 aa, synthetic construct
  REGION 1..646: note = Synthetic
  REGION 1..381: note = MISC_FEATURE - 1N3R Tau
  SITE 243: note = MISC_FEATURE - Mutation
  REGION 382..388: note = MISC_FEATURE - Furin-GSG linker
  REGION 389..410: note = MISC_FEATURE - P2A
  REGION 411..646: note = MISC_FEATURE - mCherry
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGVGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRKRRGSGATNFSLLKQAGDVEENPGPEFTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 46 - DNA, 1938 bp, synthetic construct
  misc_feature 1..1938: note = Synthetic
  misc_feature 1..1143: note = 1N3R Tau
  misc_feature 727..729: note = Mutation
  misc_feature 1144..1164: note = Furin-GSG linker
  misc_feature 1165..1230: note = P2A
  misc_feature 1231..1938: note = mCherry
  source 1..1938: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggagtcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgcggaagcgacgcggaagcggagctactaacttcagcctgctgaagcaggctggagacgtggaggagaaccctggacctgaattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 47 - protein, 412 aa, synthetic construct
  REGION 1..412: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 48 - DNA, 1236 bp, synthetic construct
  misc_feature 1..1236: note = Synthetic
  source 1..1236: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 49 - protein, 381 aa, synthetic construct
  REGION 1..381: note = Synthetic
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 50 - DNA, 1143 bp, synthetic construct
  misc_feature 1..1143: note = Synthetic
  source 1..1143: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg